(19) United States
(12) Patent Application Publication
(10) Pub. No.: US 2023/0414166 A1
(43) Pub. Date: Dec. 28, 2023

(54) METHOD AND APPARATUS FOR EVALUATING SKIN CONDITION

(71) Applicant: Panasonic Intellectual Property Management Co., Ltd., Osaka (JP)

(72) Inventors: KEIKO YUGAWA, Nara (JP); YUMIKO KATO, Osaka (JP); MOTOKI YAKO, Osaka (JP); ATSUSHI ISHIKAWA, Osaka (JP)

(21) Appl. No.: 18/464,221

(22) Filed: Sep. 10, 2023

Related U.S. Application Data
(63) Continuation of application No. PCT/JP2022/009583, filed on Mar. 7, 2022.

(30) Foreign Application Priority Data
Mar. 25, 2021 (JP) 2021-051572

(51) Int. Cl. A61B 5/00; G06T 7/00

(52) U.S. Cl. CPC A61B 5/444; A61B 5/7435; A61B 5/0077; G06T 7/0012; G06T 2207/30088

(57) ABSTRACT

A method for evaluating a user's skin condition, the method being performed by a computer, includes acquiring image data concerning part of the user's body and including information for four or more bands, determining an evaluation region in the part of the user's body in an image representing the part of the user's body in accordance with an input from the user, and generating, based on the image data, and outputting evaluation data representing an evaluation result of skin condition in the evaluation region.

### BACKGROUND

### 1. Technical Field

The present disclosure relates to a method and an apparatus for evaluating skin condition.

### 2. Description of the Related Art

The growing awareness of anti-aging has increased the importance of evaluating skin condition. This is because the evaluation of skin condition helps to improve skin condition. For example, International Publication No. 2016/080266 discloses a method for evaluating a skin blemish. In this method for evaluating a skin blemish, the regions of blemishes are extracted through image processing from the entirety of an RGB image of the skin of a user, and the number of blemishes and the areas and densities of the blemishes are quantitatively evaluated on the basis of the regions of the extracted blemishes.

In recent years, image capturing apparatuses have been developed that can acquire image information at more wavelengths than RGB images. U.S. Pat. No. 9,599,511 discloses an image capturing apparatus that uses compressed-sensing technology to obtain a hyperspectral image of an object.

### SUMMARY

One non-limiting and exemplary embodiment provides a technology for reducing the processing load in evaluation of skin condition.

In one general aspect, the techniques disclosed here feature a method for evaluating a user's skin condition, the method being performed by a computer. The method includes acquiring image data concerning part of the user's body and including information for four or more bands, determining an evaluation region in an image representing the part of the user's body in accordance with an input from the user, and generating, based on the image data, and outputting evaluation data representing an evaluation result of skin condition in the evaluation region.

A general or specific embodiment according to the present disclosure may be realized by a system, an apparatus, a method, an integrated circuit, a computer program, or a computer readable recording medium such as a recording disc or by a combination of some or all of a system, an apparatus, a method, an integrated circuit, a computer program, and a recording medium. Examples of the computer readable recording medium include nonvolatile recording media such as a compact disc read-only memory (CD-ROM), a digital versatile disc (DVD), and a Blu-ray Disc (BD). The apparatus may be formed by one or more devices. In a case where the apparatus is formed by two or more devices, the two or more devices may be arranged in one apparatus or may be arranged in two or more separate apparatuses in a divided manner. In the present specification and the claims, an “apparatus” may refer not only to one apparatus but also to a system formed by apparatuses. The apparatuses included in the “system” may include an apparatus installed at a remote place away from the other apparatuses and connected to the other apparatuses via a communication network.

According to a technology of the present disclosure, the processing load in evaluation of skin condition can be reduced.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a diagram for describing a relationship between a target wavelength range and bands included in the target wavelength range;

FIG. 1B is a diagram schematically illustrating an example of a hyperspectral image;

FIG. 2A is a diagram schematically illustrating an example of a filter array;

FIG. 2B is a diagram illustrating an example of a transmission spectrum of a first filter among filters included in the filter array illustrated in FIG. 2A;

FIG. 2C is a diagram illustrating an example of a transmission spectrum of a second filter among the filters included in the filter array illustrated in FIG. 2A;

FIG. 2D is a diagram illustrating an example of a spatial distribution of luminous transmittance of each of the bands included in the target wavelength range;

FIG. 3 is a block diagram schematically illustrating the configuration of an evaluation apparatus according to a first embodiment, which is an exemplary embodiment, of the present disclosure;

FIG. 4A is a diagram for describing an example of the procedure for registering an evaluation region and a base region in an evaluation in the first session;

FIG. 4B is a diagram for describing the example of the procedure for registering an evaluation region and a base region in an evaluation in the first session;

FIG. 4C is a diagram for describing the example of the procedure for registering an evaluation region and a base region in an evaluation in the first session;

FIG. 4D is a diagram for describing the example of the procedure for registering an evaluation region and a base region in an evaluation in the first session;

FIG. 4E is a diagram for describing the example of the procedure for registering an evaluation region and a base region in an evaluation in the first session;

FIG. 5 is a flow chart illustrating an example of an operation performed by a processing circuit in the procedure described with reference to FIGS. 4A to 4E;

FIG. 6A is a diagram for describing the procedure for displaying evaluation results in an evaluation in the first session;

FIG. 6B is a diagram for describing the procedure for displaying evaluation results in the evaluation in the first session;

FIG. 6C is a graph illustrating examples of the relationship between pixel value and wavelength for ideal skin, the most central portion, a central portion, and a peripheral portion;

FIG. 7 is a flow chart illustrating an example of an evaluation operation performed by the processing circuit in the procedure described with reference to FIGS. 6A and 6B;

FIG. 8A is a diagram for describing an example of the procedure for registering the current evaluation regions in evaluations in the second and subsequent sessions;

FIG. 8B is a diagram for describing the example of the procedure for registering the current evaluation regions in evaluations in the second and subsequent sessions;

FIG. 8C is a diagram for describing the example of the procedure for registering the current evaluation regions in evaluations in the second and subsequent sessions;

FIG. 8D is a diagram for describing the example of the procedure for registering the current evaluation regions in evaluations in the second and subsequent sessions;

FIG. 9 is a flow chart illustrating an example of an operation performed by the processing circuit in the procedure described with reference to FIGS. 8A to 8D;

FIG. 10A is a diagram for describing an example of the procedure for displaying evaluation results in the evaluations in the second and subsequent sessions;

FIG. 10B is a diagram for describing the example of the procedure for displaying evaluation results in the evaluations in the second and subsequent sessions;

FIG. 11 is a flow chart illustrating an example of an operation performed by the processing circuit in the procedure described with reference to FIGS. 10A and 10B;

FIG. 12A is a diagram schematically illustrating an example of a full-reconstruction table stored in a storage device;

FIG. 12B is a diagram schematically illustrating an example of partial-reconstruction tables stored in the storage device;

FIG. 12C is a diagram schematically illustrating an example of a table of registered regions stored in the storage device in the first session;

FIG. 12D is a diagram schematically illustrating an example of tables of evaluation results stored in the storage device;

FIG. 13 is a diagram schematically illustrating the configuration of an evaluation apparatus, which is an exemplary modification of the first embodiment;

FIG. 14 is a block diagram schematically illustrating an example of an evaluation system according to a second embodiment;

FIG. 15 is a sequence diagram illustrating an operation performed in the first session between an evaluation apparatus and a server according to the second embodiment;

FIG. 16 is a sequence diagram illustrating an operation performed in the second and subsequent sessions between the evaluation apparatus and the server according to the second embodiment; and

FIG. 17 is a diagram schematically illustrating the configuration of an evaluation system, which is an exemplary modification of the second embodiment.

### DETAILED DESCRIPTIONS

In the present disclosure, all or some of circuits, units, devices, members, or portions or all or some of the functional blocks of a block diagram may be executed by, for example, one or more electronic circuits including a semiconductor device, a semiconductor integrated circuit (IC), or a large-scale integration circuit (LSI). The LSI or the IC may be integrated onto one chip or may be formed by combining chips. For example, functional blocks other than a storage device may be integrated onto one chip. In this case, the term LSI or IC is used; however, the term to be used may change depending on the degree of integration, and the term “system LSI”, “very large-scale integration (VLSI)”, or “ultra-large-scale integration (ULSI)” may be used. A field-programmable gate array (FPGA) or a reconfigurable logic device that allows reconfiguration of interconnection inside an LSI or setup of a circuit section inside an LSI can also be used for the same purpose, the FPGA and the reconfigurable logic device being programmed after the LSIs are manufactured.

Furthermore, functions or operations of all or some of the circuits, the units, the devices, the members, or the portions can be executed through software processing. In this case, software is recorded in one or more non-transitory recording mediums such as a read-only memory (ROM), an optical disc, or a hard disk drive, and when the software is executed by a processing device (a processor), the function specified by the software is executed by the processing device (the processor) and peripheral devices. The system or the apparatus may have the one or more non-transitory recording mediums in which the software is recorded, the processing device (the processor), and a hardware device to be needed such as an interface.

In the following, exemplary embodiments of the present disclosure will be described. Note that any one of embodiments to be described below is intended to represent a general or specific example. Numerical values, shapes, constituent elements, arrangement positions and connection forms of the constituent elements, steps, and the order of steps are examples, and are not intended to limit the present disclosure. Among the constituent elements of the following embodiments, constituent elements that are not described in independent claims representing the most generic concept are described as optional constituent elements. Each drawing is a schematic diagram and is not necessarily precisely illustrated. Furthermore, in each drawing, substantially the same or similar constituent elements are denoted by the same reference signs. Redundant description may be omitted or simplified.

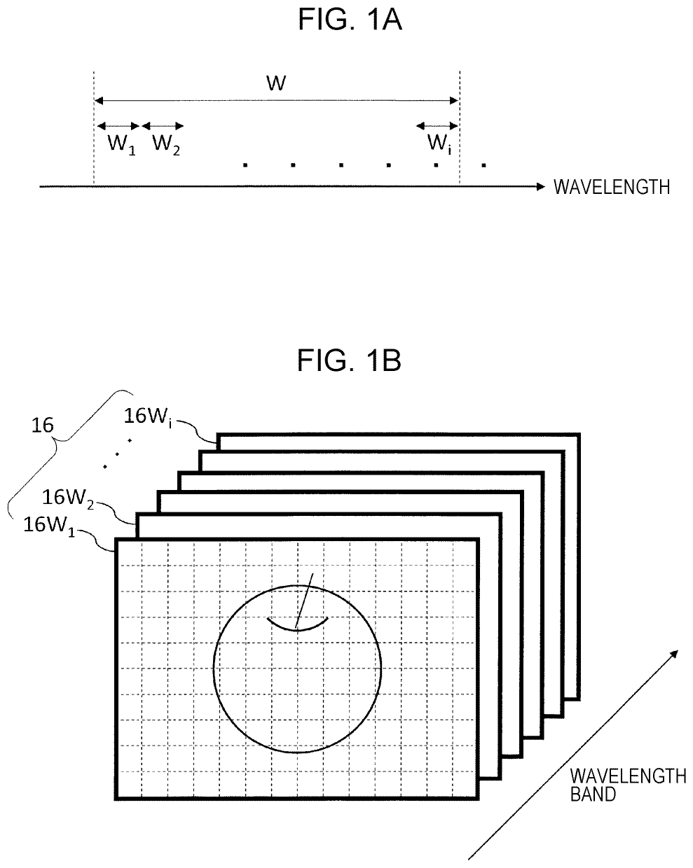

First, an example of a hyperspectral image will be briefly described with reference to FIGS. 1A and 1B. A hyperspectral image is image data having more wavelength information than a typical RGB image. Pixels of an RGB image each have values for three bands, which are red (R), green (G), and blue (B). In contrast, pixels of a hyperspectral image each have values for a greater number of bands than those of an RGB image. In this specification, a “hyperspectral image” refers to image data in which pixels each have values for four or more bands included in a predetermined target wavelength band. A value that each pixel has on a band basis will be referred to as a “pixel value” in the following description. The number of bands in a hyperspectral image is typically greater than or equal to 10 and may exceed 100 in some cases. A “hyperspectral image” may also be referred to as a “hyperspectral data cube” or a “hyperspectral cube”.

FIG. 1A is a diagram for describing a relationship between a target wavelength range W and bands W₁, W₂, . . . , Wᵢ included in the target wavelength range W. The target wavelength range W may be set to various ranges depending on applications. The target wavelength range W may have, for example, a wavelength range of visible light of about 400 nm to about 700 nm, a wavelength range of near-infrared rays of about 700 nm to about 2500 nm, or a wavelength range of near-ultraviolet rays of about 10 nm to about 400 nm. Alternatively, the target wavelength range W may be the mid-infrared wavelength range or the far-infrared wavelength range. In this manner, a wavelength region used is not limited to the visible light region. In this specification, not only visible light but also electromagnetic waves having wavelengths outside the wavelength range of visible light will be referred to as “light” for convenience’ sake. Examples of the electromagnetic waves include ultraviolet rays and near-infrared rays.

In the example illustrated in FIG. 1A, i is set to any integer greater than or equal to 4, the target wavelength range W is equally divided into i wavelength regions, and the i wavelength regions are referred to as a band W₁, a band W₂, . . . , and a band Wᵢ. Note that the example is not limited to this one. The bands included in the target wavelength range W may be freely set. For example, the bands may have different widths. There may be a gap between adjacent bands among the bands. In a case where there are four or more bands, more information can be acquired from a hyperspectral image than from an RGB image.

FIG. 1B is a diagram schematically illustrating an example of a hyperspectral image 16. In the example illustrated in FIG. 1B, an imaging target is an apple. The hyperspectral image 16 includes an image 16W₁ for the band W₁, an image 16W₂ for the band W₂, . . . , and an image 16Wᵢ for the band Wᵢ. Each of these images include pixels arranged two-dimensionally. FIG. 1B illustrates vertical broken lines and horizontal broken lines to represent borders between the pixels. The actual number of pixels per image may be a large value such as several tens of thousands to several tens of millions; however, in FIG. 1B, the borders between the pixels are illustrated supposing that the number of pixels is extremely small for convenience’ sake. An image sensor has light detection devices, and each light detection device is configured to detect reflected light caused when an object is irradiated with light. For each light detection device, a signal indicating the amount of light detected by the light detection device represents a pixel value of a pixel corresponding to the light detection device. Each pixel of the hyperspectral image 16 has a pixel value for each band. Thus, information regarding a two-dimensional distribution of the spectrum of the object can be obtained by acquiring the hyperspectral image 16. On the basis of the spectrum of the object, optical characteristics of the object can be correctly analyzed.

Next, an example of a method for generating a hyperspectral image will be briefly described. A hyperspectral image can be acquired through imaging performed using, for example, a spectroscopic element such as a prism or a grating. In a case where a prism is used, when reflected light or transmitted light from an object passes through the prism, the light is emitted from a light emission surface of the prism at an emission angle corresponding to the wavelength of the light. In a case where a grating is used, when reflected light or transmitted light from the object is incident on the grating, the light is diffracted at a diffraction angle corresponding to the wavelength of the light. A hyperspectral image can be obtained by separating, using a prism or a grating, light from the object into bands and detecting the separated light on a band basis.

A hyperspectral image can also be acquired using a compressed-sensing technology disclosed in U.S. Pat. No. 9,599,511. In the compressed-sensing technology disclosed in U.S. Pat. No. 9,599,511, light that has passed through a filter array referred to as an encoder and is then reflected by an object is detected by an image sensor. The filter array includes filters arranged two-dimensionally. These filters have transmission spectra unique thereto in a respective manner. Through imaging using such a filter array, one two-dimensional image into which image information regarding bands is compressed is obtained as a compressed image. In the compressed image, the spectrum information regarding the object is compressed and recorded as one pixel value per pixel.

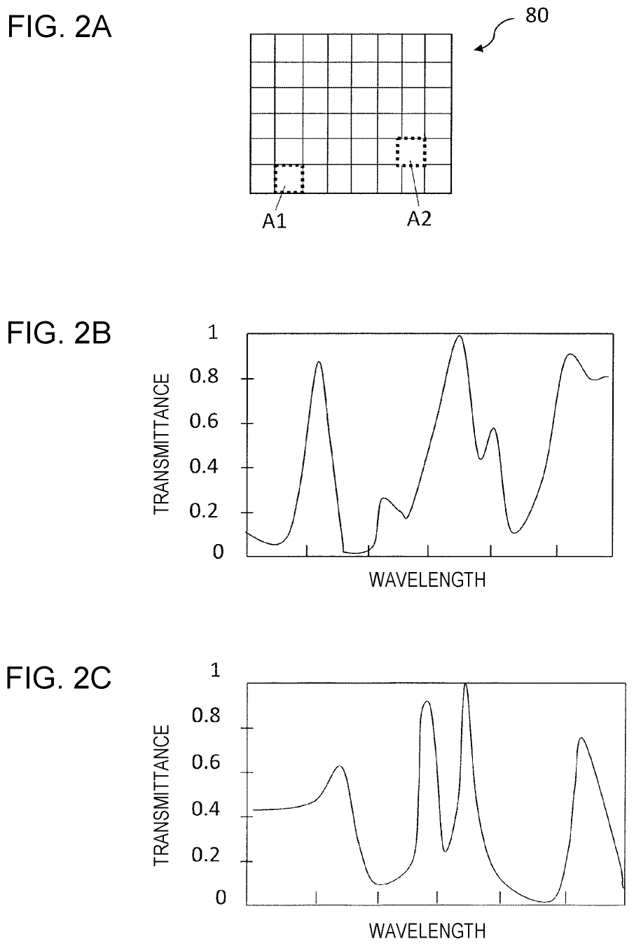

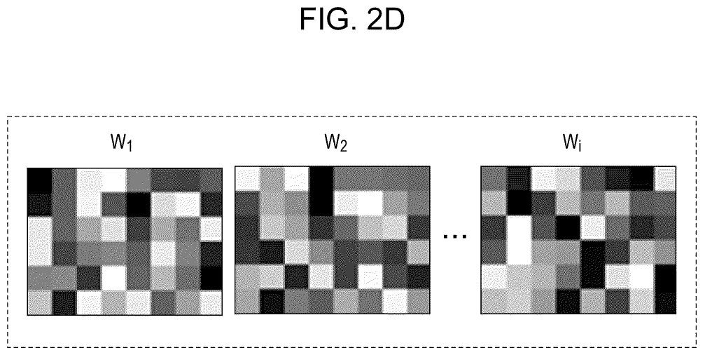

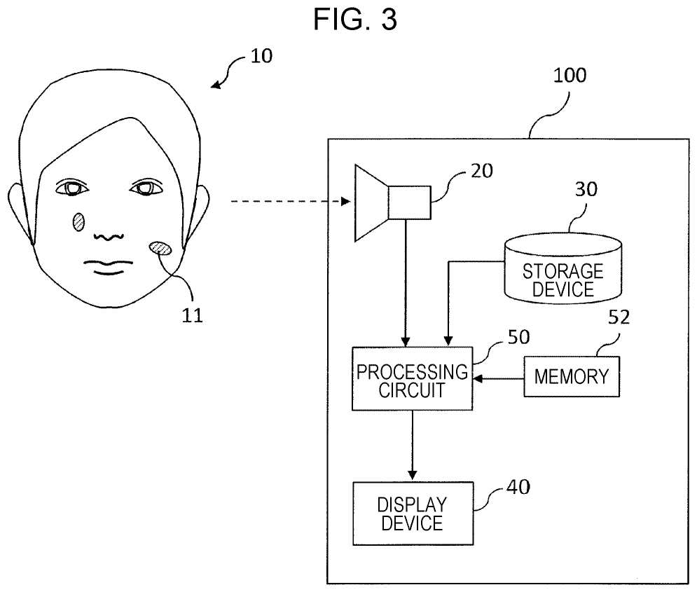

FIG. 2A is a diagram schematically illustrating an example of a filter array 80. The filter array 80 includes filters arranged two-dimensionally. Each filter has a transmission spectrum set individually. The transmission spectrum is expressed by a function T(λ), where the wavelength of incident light is λ. The transmission spectrum T(λ) may have a value greater than or equal to 0 and less than or equal to 1. In the example illustrated in FIG. 2A, the filter array 80 has 48 rectangular filters arranged in 6 rows and 8 columns. This is merely an example, and a larger number of filters than this may be provided in actual applications. The number of filters included in the filter array 80 may be about the same as, for example, the number of pixels of the image sensor.

FIGS. 2B and 2C illustrate the transmission spectrum of a first filter A1 and that of a second filter A2, respectively, among the filters included in the filter array 80 in FIG. 2A. The transmission spectrum of the first filter A1 and that of the second filter A2 are different from each other. In this manner, the transmission spectra of the filters of the filter array 80 are different from each other. Note that the transmission spectra of all the filters are not necessarily different from each other. The transmission spectra of at least two or more filters among the filters are different from each other in the filter array 80. That is, the filter array 80 includes two or more filters that have different transmission spectra from each other. In one example, the number of patterns of the transmission spectra of the filters included in the filter array 80 may be the same as i, which is the number of bands included in the target wavelength range, or greater than or equal to i. The filter array 80 may be designed such that more than half of the filters have different transmission spectra from each other.

FIG. 2D is a diagram illustrating an example of a spatial distribution of luminous transmittance of each of the bands W₁, W₂, . . . , Wᵢ included in the target wavelength range. In the example illustrated in FIG. 2D, differences in shading between the filters represent differences in luminous transmittance. The lighter the shade of the filter, the higher the transmittance. The darker the shade of the filter, the lower the transmittance. As illustrated in FIG. 2D, the spatial distribution of luminous transmittance differs from band to band.

A hyperspectral image can be reconstructed from a compressed image using data representing the spatial distribution of luminous transmittance of each band of the filter array. For reconstruction, compressed-sensing technology is used. The data used in reconstruction processing and representing the spatial distribution of luminous transmittance of each band of the filter array is referred to as a “reconstruction table”. In the compressed-sensing technology, a prism or a grating does not need to be used, and thus a hyperspectral camera can be miniaturized. Furthermore, in the compressed-sensing technology, the amount of data processed by a processing circuit can be reduced by using a compressed image.

Next, a method for reconstructing a hyperspectral image from a compressed image using a reconstruction table will be described. Compressed image data g acquired by the image sensor, a reconstruction table H, and hyperspectral image data f satisfy Eq. (1) below.

g=Hf (1)

In this case, the compressed image data g and the hyperspectral image data f constitute vector data, and the reconstruction table H is matrix data. When the number of pixels of the compressed image data g is denoted by N_{g}, the compressed image data g is expressed as a one-dimensional array, that is, a vector having N_{g} elements. When the number of pixels of the hyperspectral image data f is denoted by N_{f}, and the number of bands is denoted by M, the hyperspectral image data f is expressed as a one-dimensional array, that is, a vector having (N_{f}×M) elements. The reconstruction table H is expressed as a matrix having elements of N_{g} rows and (N_{f}×M) columns. N_{g} and N_{f} can be designed to have the same value.

When the vector g and the matrix H are given, it seems that f can be calculated by solving an inverse problem of Eq. (1). However, the number of elements (N_{f}×M) of the data f to be obtained is greater than the number of elements N_{g} of the acquired data g, and thus this problem is an ill-posed problem, and the problem cannot be solved as it is. Thus, the redundancy of the images included in the data f is used to obtain a solution using a compressed-sensing method. Specifically, the data f to be obtained is estimated by solving the following Eq. (2).

$${f}^{′} = {arg min}_{f} \{ {‖ g - Hf ‖}_{{l}_{2}} + τϕ ( f ) \} \qquad ( 2 )$$

In this case, f denotes estimated data of the data f. The first term in the braces of the equation above represents a shift between an estimation result Hf and the acquired data g, which is a so-called residual term. In this case, the sum of squares is treated as the residual term; however, an absolute value, a root-sum-square value, or the like may be treated as the residual term. The second term in the braces is a regularization term or a stabilization term, which will be described later. Eq. (2) means to obtain f that minimizes the sum of the first term and the second term. The processing circuit can cause a solution to converge through a recursive iterative operation and can calculate the final solution f.

The first term in the braces of Eq. (2) refers to a calculation for obtaining the sum of squares of the differences between the acquired data g and Hf, which is obtained by performing a system conversion on f in the estimation process using the matrix H. The second term Φ(f) is a constraint for regularization of f and is a function that reflects sparse information regarding the estimated data. This function provides an effect in that estimated data is smoothed or stabilized. The regularization term can be expressed using, for example, discrete cosine transformation (DCT), wavelet transform, Fourier transform, or total variation (TV) of f. For example, in a case where total variation is used, stabilized estimated data can be acquired in which the effect of noise of the data g, observation data, is suppressed. The sparsity of the object in a space of each regularization term differs with the texture of the object. A regularization term having a regularization term space in which the texture of the object becomes sparser may be selected. Alternatively, regularization terms may be included in calculation. T is a weighting factor. The greater the weighting factor r, the greater the amount of reduction of redundant data, thereby increasing a compression rate. The smaller the weighting factor r, the lower the convergence to the solution. The weighting factor T is set to an appropriate value with which f is converged to a certain degree and is not compressed too much.

A more detailed method for acquiring a hyperspectral image using a compressed-sensing technology is disclosed in U.S. Pat. No. 9,599,511. The entirety of the disclosed content of U.S. Pat. No. 9,599,511 is incorporated herein by reference. Note that a method for acquiring a hyperspectral image through imaging is not limited to the above-described method using compressed sensing. For example, a hyperspectral image may be acquired through imaging using a filter array in which pixel regions including four or more filters having different transmission wavelength ranges from each other are arranged two-dimensionally. Alternatively, a hyperspectral image may be acquired using a spectroscopic method using a prism or a grating.

A hyperspectral camera can evaluate skin condition more accurately than a typical RGB camera. In contrast, hyperspectral image data may cause a high processing load because it contains image information for many bands. By using a method for evaluating skin condition according to an embodiment of the present disclosure, such a processing load can be reduced. In a method according to an embodiment of the present disclosure, skin condition in an evaluation region of a part of a user's body is evaluated on the basis of image data concerning the part of the user's body. The image data includes information for four or more bands. Such image data may be, for example, compressed image data or hyperspectral image data. The evaluation region is determined in accordance with an input from the user. Unlike in a method described in International Publication No. 2016/080266, the entirety of image data needs not be processed to extract a specific region in the method according to the present embodiment. As a result, it is possible to reduce the processing load in evaluation of skin condition. In the following, a method and an apparatus for evaluating skin condition according to embodiments of the present disclosure will be described.

A method according to a first aspect is a method for evaluating a user's skin condition, the method being performed by a computer. The method includes acquiring image data concerning part of the user's body and including information for four or more bands, determining an evaluation region in an image representing the part of the user's body in accordance with an input from the user, and generating, based on the image data, and outputting evaluation data representing an evaluation result of skin condition in the evaluation region.

By using this method, it is possible to reduce the processing load in the evaluation of skin condition.

By using the method, evaluation data representing an evaluation result of skin condition in only the evaluation region may be generated, on the basis of the image data, and output. As a result, a target for which evaluation data is to be generated can be narrowed down, and the processing load in evaluation of skin condition can further be reduced.

Moreover, in the method, the evaluation data may exclude an evaluation result of skin in a different region from the evaluation region. As a result, a target for which evaluation data is to be generated can be narrowed down, and the processing load in evaluation of skin condition can further be reduced.

A method according to a second aspect is the method according to the first aspect that further includes determining a base region located at a different position from the evaluation region in the image. The evaluation result includes a comparison result between the skin condition in the evaluation region and skin condition in the base region.

By using this method, the skin conditions in two different regions can be compared with each other.

A method according to a third aspect is the method according to the first aspect that further includes treating the skin condition in the evaluation region as current skin condition in the evaluation region, and acquiring data indicating past skin condition in the evaluation region. The evaluation result includes a comparison result between the current skin condition in the evaluation region and the past skin condition in the evaluation region.

By using this method, the current and past skin conditions in the evaluation region can be compared with each other.

A method according to a fourth aspect is the method according to any one of the first to third aspects in which the acquiring the image data includes acquiring compressed image data. The compressed image data is obtained by compressing image information regarding the part of the user's body for the four or more bands into one image.

By using this method, the amount of data in processing of image data can be reduced.

A method according to a fifth aspect is the method according to the fourth aspect that further includes generating, for the evaluation region, partial-image data corresponding to at least one band among the four or more bands from the image data. The generating and outputting includes generating, based on the partial-image data, and outputting the evaluation data.

By using this method, the processing load can be reduced.

A method according to a sixth aspect is the method according to the fifth aspect in which the compressed image data is acquired by imaging the part of the user's body through a filter array. The filter array has filters arranged two-dimensionally. Transmission spectra of at least two or more filters among the filters are different from each other. The generating the partial-image data includes generating the partial-image data using at least one reconstruction table corresponding to the at least one band. The reconstruction table represents a spatial distribution of luminous transmittance of each band for the filter array in the evaluation region.

By using this method, partial-image data can be generated.

A method according to a seventh aspect is the method according to any one of the first to sixth aspects that further includes causing a display device to display a graphical user interface for the user to specify the evaluation region.

By using this method, the user can specify an evaluation region through the graphical user interface.

A method according to an eighth aspect is the method according to the seventh aspect in which the graphical user interface displays the image representing the part of the user's body.

By using this method, the user can specify an evaluation region while viewing an image representing part of his or her body.

A method according to a ninth aspect is the method according to any one of the first to eighth aspects in which the image includes information for one or more, but no more than three, bands.

By using this method, a monochrome image or an RGB image can be used, for example, as an image representing the part of the user's body.

A method according to a tenth aspect is the method according to the ninth aspect in which the image is generated based on the image data.

By using this method, a monochrome image or an RGB image, for example, can be generated from compressed image data or hyperspectral image data.

A method according to an eleventh aspect is the method according to the ninth aspect in which the image data is treated as first image data and that further includes acquiring second image data concerning the part of the user's body and including information for one or more, but no more than three, bands. The image is an image represented by the second image data.

By using this method, the processing load can be reduced by separately acquiring, for example, a monochrome image or an RGB image representing the part of the user's body.

A method according to a twelfth aspect is the method according to any one of the first to eleventh aspects in which the skin condition is a state of a blemish.

By using this method, the state of the blemish can be evaluated.

A processing apparatus according to a thirteenth aspect includes a processor, and a memory in which a computer program that the processor executes is stored. The computer program causes the processor to perform acquiring image data concerning part of a user's body and including information for four or more bands, determining an evaluation region in the part of the user's body in an image representing the part of the user's body in accordance with an input from the user, and generating, based on the image data, and outputting evaluation data representing an evaluation result of skin condition in the evaluation region.

In this processing apparatus, the processing load in the evaluation of skin condition can be reduced.

A computer program according to a fourteenth aspect causes a computer to perform acquiring image data concerning part of a user's body and including information for four or more bands, determining an evaluation region in the part of the user's body in an image representing the part of the user's body in accordance with an input from the user, and generating, based on the image data, and outputting evaluation data representing an evaluation result of skin condition in the evaluation region.

With this computer program, the processing load in the evaluation of skin condition can be reduced.

### EMBODIMENTS

### Evaluation Apparatus

First, with reference to FIG. 3, an example of an evaluation apparatus according to a first embodiment of the present disclosure will be described. The evaluation apparatus evaluates a user's skin condition. In the evaluation apparatus, a hyperspectral camera using a compressed-sensing technology is used to evaluate skin condition; however, a hyperspectral camera that does not use a compressed-sensing technology may be used. In the description below, the skin of a user's face is exemplified as the skin of part of the user's body, and the states of blemishes are exemplified as skin condition. The part of the user's body may be any part of the user's body with skin, such as his or her arm or leg, other than his or her face. Skin condition may be related to wrinkles or acne condition, or skin moisture or sebum content, for example, in addition to blemishes.

FIG. 3 is a block diagram schematically illustrating the configuration of an evaluation apparatus 100 according to the first embodiment, which is an exemplary embodiment of the present disclosure. FIG. 3 illustrates a user's face 10 viewed from the front. The face 10 has a blemish 11 to the right of the nose and another blemish 11 on the left cheek. The evaluation apparatus 100 illustrated in FIG. 3 includes a hyperspectral camera 20, a storage device 30, a display device 40, a processing circuit 50, and a memory 52. The processing circuit 50 controls the hyperspectral camera 20, the storage device 30, and the display device 40. The configuration of the evaluation apparatus 100 illustrated in FIG. 3 may be, for example, part of the configuration of a mobile terminal, such as a smartphone, or a personal computer. Alternatively, the evaluation apparatus 100 illustrated in FIG. 3 may be a dedicated apparatus that evaluates skin condition.

The face 10 is irradiated with light emitted from a light source for evaluation or ambient light. Light emitted from the light source for evaluation or ambient light may include, for example, visible light or may include visible light and near-infrared rays.

The hyperspectral camera 20 captures an image of the face 10 by detecting reflected light generated by the face 10 as a result of light irradiation. A dashed arrow illustrated in FIG. 3 represents reflected light generated by the face 10. The hyperspectral camera 20 may have the above-described light source for evaluation. The hyperspectral camera 20 generates and outputs compressed image data of the face 10. The hyperspectral camera 20 may be attached to the evaluation apparatus 100 externally.

The storage device 30 stores a reconstruction table for a filter array used in compressed-sensing technology and data generated in the process of evaluating skin condition. The reconstruction table is a reconstruction table corresponding to all bands for the entire region. Alternatively, the reconstruction table is a reconstruction table corresponding to some of the bands for the entire region, a reconstruction table corresponding to all bands for part of the region, or a reconstruction table corresponding to some of the bands for part of the region. In the following description, the reconstruction table corresponding to all bands for the entire region among the above-described reconstruction tables is referred to as a “full-reconstruction table”, and the other reconstruction tables are referred to as “partial-reconstruction tables”. The storage device 30 includes, for example, any storage medium such as a semiconductor memory, a magnetic storage device, or an optical storage device.

The processing circuit 50 acquires compressed image data from the hyperspectral camera 20 and acquires the reconstruction table from the storage device 30. The processing circuit 50 generates, on the basis of these acquired data, partial-image data corresponding to at least one band for a partial region of the face 10. “Partial-image data” refers to part of hyperspectral image data. The hyperspectral image data has three-dimensional image information based on a two-dimensional space and wavelengths. “Part” may be part of the space or part of a wavelength axis. The processing circuit 50 evaluates, on the basis of the partial-image data, skin condition in the partial region of the face 10 and causes the display device 40 to display an evaluation result. The partial region may be, for example, a blemish 11 on the face 10. Details of an evaluation method will be described below.

Regarding generation of partial-image data, the processing circuit 50 may reconstruct hyperspectral image data from compressed image data using the full-reconstruction table and extract the above-described partial-image data from the hyperspectral image data. Alternatively, the processing circuit 50 may generate a partial-reconstruction table from the full-reconstruction table and generate, using the partial-reconstruction table, partial-image data from compressed image data in a selective manner. The partial-reconstruction table includes a luminous transmittance for each pixel for at least one band and a luminous transmittance for each pixel for a band obtained by combining the other bands in a partial region. The luminous transmittance for each pixel for the band obtained by combining the other bands is a luminous transmittance obtained by adding the luminous transmittances of the other bands or the average of the obtained luminous transmittances. Generation of the partial-image data in a selective manner can reduce the processing load, compared with reconstruction of the hyperspectral image data. Details of this selective generation method are disclosed in Japanese Patent Application No. 2020-056353.

As described below, in a case where RGB image data is to be generated from compressed image data, the processing circuit 50 may reconstruct hyperspectral image data from compressed image data and generate RGB image data from the hyperspectral image data. Alternatively, the processing circuit 50 may generate a partial-reconstruction table corresponding to bands for RGB from the full-reconstruction table and generate, using the partial-reconstruction table, RGB image data from the compressed image data in a selective manner.

A computer program that the processing circuit 50 executes is stored in the memory 52 such as a read-only memory (ROM) or a random access memory (RAM). In this specification, an apparatus that includes the processing circuit 50 and the memory 52 is also referred to as a “processing apparatus”. The processing circuit 50 and the memory 52 may be integrated on a single circuit board or may be provided on separate circuit boards.

The display device 40 displays a graphical user interface (GUI) for the user to specify a region to be used to evaluate skin condition from within the face 10. Furthermore, the display device 40 displays evaluation results. The display device 40 may be, for example, a display of a mobile terminal or a personal computer.

The evaluation apparatus 100 may further include a gyroscope to compensate for camera shake during image capturing in addition to the above-described configuration and may further include an output device for instructing the user to perform image capturing. The output device may be, for example, a speaker or a vibrator.

A skin condition evaluation method according to the first embodiment is different between the first session and the second and subsequent sessions. In an evaluation in the first session, regions with blemishes of concern and an ideal region without blemishes in the face 10 are registered. In the following description, a region with a blemish of concern is referred to as an “evaluation region”, and an ideal region without blemishes is referred to as a “base region”. The evaluation regions and the base region are located at different positions from each other. The number of evaluation regions may be greater than or equal to one, and the number of base regions may be greater than or equal to one. After registering the evaluation regions and the base region, skin condition in the evaluation regions is evaluated. In evaluations in the second and subsequent sessions, the current evaluation regions are determined on the basis of the evaluation regions registered in the first session, and skin condition in the evaluation regions is evaluated. Skin condition may be evaluated on a daily, weekly, monthly, or yearly cycle, for example. Since the skin turnover cycle is about 28 days, skin condition may be evaluated on the basis of this cycle. Evaluation Method in First Session

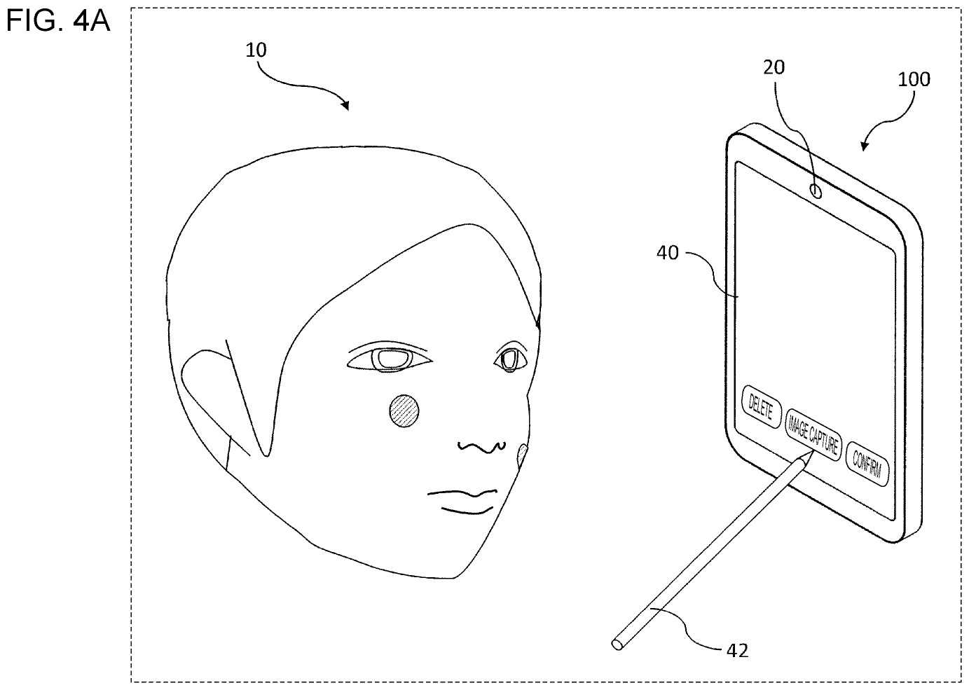

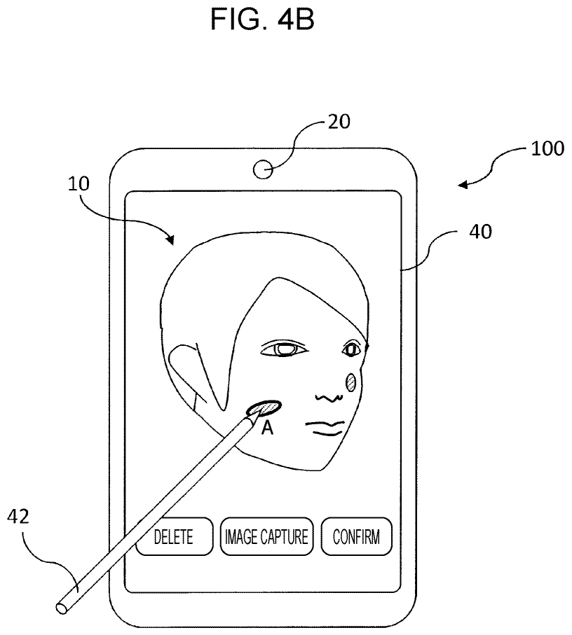

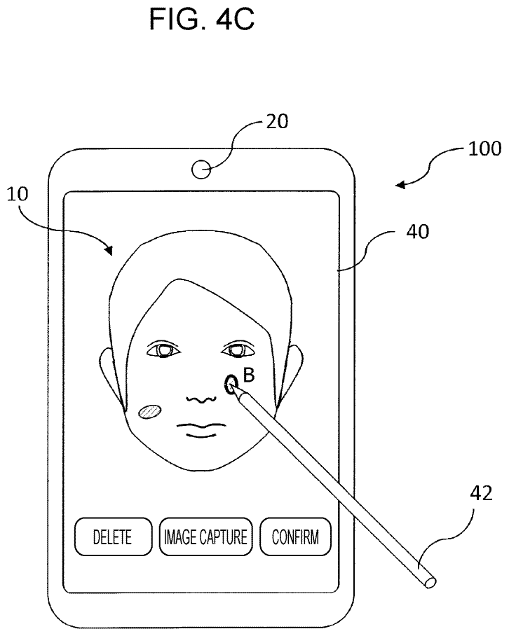

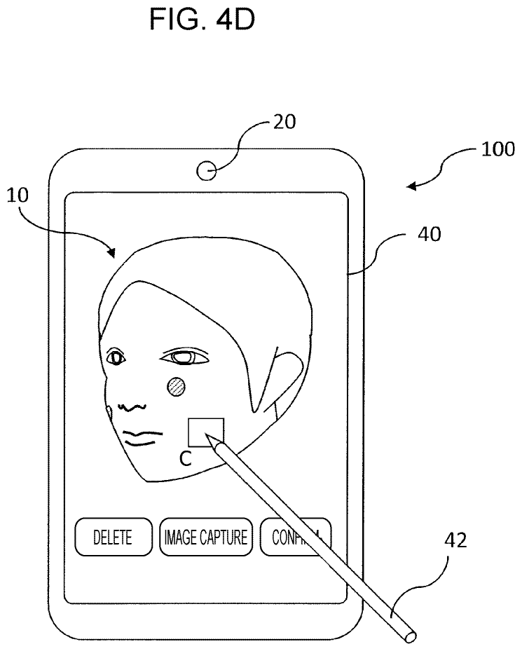

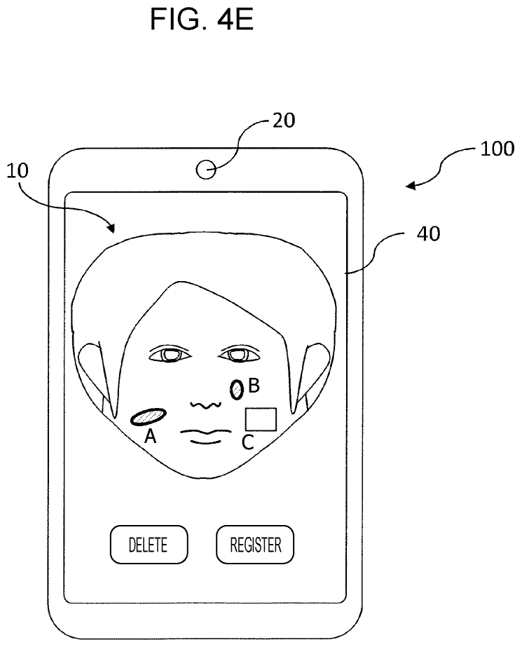

In the following, an evaluation method in the first session will be described with reference to FIGS. 4A to 7. FIGS. 4A to 4E are diagrams for describing an example of the procedure for registering an evaluation region and a base region in an evaluation in the first session. In the example illustrated in FIGS. 4A to 4E, the evaluation apparatus 100 is a smartphone, and the display device 40 is the display of the smartphone. An application for performing the method according to the first embodiment is installed in the memory 52 of the evaluation apparatus 100. In the following description, “the display device 40 displays . . . ” means that “the display device 40 displays a GUI that presents . . . ”.

When the application is started, the processing circuit 50 causes a speaker to output the following audio. The audio is, for example, “Please capture images of your face continuously from the left side to the right side. Please observe the captured images of the face, and if there is a blemish of concern, touch the blemish with a touch pen. Furthermore, please press and hold the touch pen on ideal skin without blemishes”. The processing circuit 50 acquires data indicating the date and time at which an operation was started for evaluation. In the following description, the date and time is referred to as a “start date and time”.

As illustrated in FIG. 4A, with the hyperspectral camera 20 of the evaluation apparatus 100 oriented so as to face the face 10 from the front left, the user touches an image capture button displayed by the display device 40 with a touch pen 42. The touch pen 42 is an example of a pointing device. The processing circuit 50 receives an image capturing signal and causes the hyperspectral camera 20 to capture an image of the face 10. The hyperspectral camera 20 generates compressed image data of the face 10. The processing circuit 50 generates, using the reconstruction table, RGB image data of the face 10 from the compressed image data. The processing circuit 50 causes the display device 40 to display an RGB image represented by the RGB image data. Not an RGB image but a monochrome image may be displayed.

As illustrated in FIG. 4B, the display device 40 displays a captured image of the left side of the face 10. A mirror image of the face 10 is reversed left to right from the face 10 illustrated in FIG. 3. The user observes the captured image of the left side of the face 10 and touches, with the touch pen 42, a region near the center of a blemish of concern on the left cheek of the face 10 as illustrated in FIG. 4B. The processing circuit 50 receives a touch signal, extracts the region of the blemish through edge detection based on the position that is specified with a touch, determines the region to be an evaluation region, and assigns a label such as “A” to the evaluation region, for example. In this manner, the evaluation region A is determined in the image that represents the face 10. The processing circuit 50 causes the display device 40 to display the edge and label of the evaluation region as illustrated in FIG. 4B. The bold ellipse illustrated in FIG. 4B represents the edge.

Next, with the hyperspectral camera 20 of the evaluation apparatus 100 oriented so as to face the face 10 from the front, the user touches the image capture button displayed by the display device 40 with the touch pen 42. As illustrated in FIG. 4C, the display device 40 displays a captured image of the front side of the face 10. The user observes the captured image of the front side of the face 10 and touches a region near the center of a blemish of concern to the right of the nose of the face 10 with the touch pen 42 as illustrated in FIG. 4C. The processing circuit 50 receives a touch signal, extracts the region of the blemish through edge detection based on the position that is specified with a touch, determines the region to be an evaluation region, and assigns a label such as “B” to the evaluation region, for example. In this manner, the evaluation region B is determined in the image that represents the face 10. The processing circuit 50 causes the display device 40 to display the edge and label of the evaluation region as illustrated in FIG. 4C.

Next, with the hyperspectral camera 20 of the evaluation apparatus 100 oriented so as to face the face 10 from the front right, the user touches the image capture button displayed by the display device 40 with the touch pen 42. As illustrated in FIG. 4D, the display device 40 displays a captured image of the right side of the face 10. The user observes the captured image of the right side of the face 10 and presses and holds, with the touch pen 42, a region of ideal skin without blemishes on the right side of the face 10 as illustrated in FIG. 4D. The processing circuit 50 receives a press-and-hold signal, determines a rectangular region having a certain area including the position that is specified with a long press to be a base region, which is an ideal skin region, and assigns a label such as “C” to the base region, for example. In this manner, the base region C is determined in the image that represents the face 10. The processing circuit 50 causes the display device 40 to display the perimeter and label of the base region as illustrated in FIG. 4D.

In the above-described example, the images of the face 10 are captured from three angles: the front left, the front, and the front right. Images of the face 10 may be captured from many more different angles from left to right, or from above or below.

As illustrated in FIGS. 4A to 4D, the display device 40 displays a delete button and a confirm button in addition to the image capture button. The delete button is a button for canceling selection of the evaluation or base region. The confirm button is a button for confirming all the selected evaluation and base regions.

In a case where the user touches the confirm button, the processing circuit 50 receives a confirmation signal and causes the display device 40 to display a two-dimensional composite image obtained by connecting the images of the left, front, and right sides of the face 10 as illustrated in FIG. 4E. On the composite image, the edges and labels of the evaluation regions and the perimeter and label of the base region are superposed to indicate the evaluation regions and the base region. Instead of the composite image, the left, front, and right side images may be separately displayed, or a three-dimensional image may be displayed. On the composite image, the evaluation and base regions may be specified using, for example, the following facial coordinate system. In the facial coordinate system, the axis that passes through the tails of the user's eyes is the X axis, the axis that passes through the bridge of the user's nose is the Y axis, and the point where the X and Y axes intersect is the origin.

As illustrated in FIG. 4E, the display device 40 displays a delete button and a register button. The delete button is a button for respecifying evaluation and base regions. The register button is a button for registering a composite image and the evaluation and base regions. In a case where the user selects the register button, the processing circuit 50 receives a registration signal and causes the storage device 30 to store data to be used in an evaluation in the first session. The data may include, for example, data indicating the start date and time of the first session, compressed image data of the left, front, and right sides of the face 10, data representing the composite image, and data indicating the evaluation and base regions. The stored evaluation and base regions are also referred to as “registered regions”.

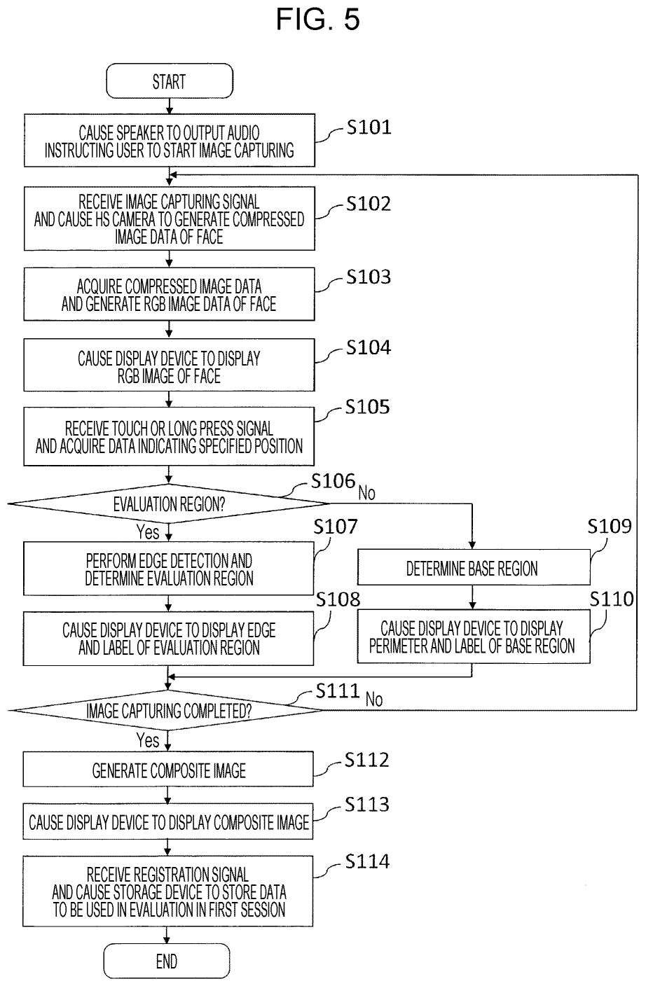

FIG. 5 is a flow chart illustrating an example of an operation performed by the processing circuit 50 in the procedure described with reference to FIGS. 4A to 4E. An “HS camera” described in FIG. 5 means a hyperspectral camera. The same applies to the following drawings. The processing circuit 50 performs the operations of Steps S101 to S114 below. The steps included in the flow chart illustrated in FIG. 5 may be reordered as long as there is no inconsistency, and may include additional other steps. The same applies to the steps included in the flow charts illustrated in the other drawings.

### Step S101

The processing circuit 50 causes the speaker to output audio instructing the user to start image capturing. The processing circuit 50 acquires data indicating the start date and time of the first session.

### Step S102

The processing circuit 50 receives an image capturing signal and causes the hyperspectral camera 20 to capture an image of the face 10. The hyperspectral camera 20 generates and outputs compressed image data of the face 10.

### Step S103

The processing circuit 50 acquires the compressed image data and generates RGB image data of the face 10 using the reconstruction table.

### Step S104

The processing circuit 50 causes the display device 40 to display an RGB image based on the RGB image data.

### Step S105

The processing circuit 50 receives a touch or long press signal and acquires data indicating the specified position.

### Step S106

The processing circuit 50 determines whether the received signal indicates an evaluation region. When receiving a touch signal, the processing circuit 50 can determine that the signal indicates an evaluation region. When receiving a long press signal, the processing circuit 50 can determine that the signal indicates a base region. When Yes in Step 106, the processing circuit 50 performs the operation of Step S107. When No in Step 106, the processing circuit performs the operation of Step S109.

### Step S107

The processing circuit 50 performs edge detection to extract the region of a blemish and determines the region to be an evaluation region.

### Step S108

The processing circuit 50 causes the display device 40 to display the edge and label of the evaluation region.

### Step S109

The processing circuit 50 determines a rectangular region having a certain area including the specified position to be a base region. The rectangular region having a certain area is, for example, a region that is centered around the point that is subjected to a long press and in which m pixels are arranged vertically, and n pixels are arranged horizontally. The values of m and n are arbitrary.

### Step S110

The processing circuit 50 causes the display device 40 to display the perimeter and label of the base region.

### Step S111

The processing circuit 50 determines whether image capturing is completed. In a case where the processing circuit 50 receives a completion signal, the processing circuit 50 can determine that image capturing is completed. In a case where the processing circuit 50 does not receive a confirmation signal within a certain period of time, the processing circuit 50 can determine that image capturing is not completed. When Yes in Step S111, the processing circuit 50 performs the operation of Step S112. When No in Step S111, the processing circuit performs the operation of Step S102 again.

### Step S112

The processing circuit 50 generates a composite image by connecting images of the left, front, and right sides of the face 10. On the composite image, the edges and labels of the evaluation regions and the perimeter and label of the base region are superposed to indicate the registered regions.

### Step S113

The processing circuit 50 causes the display device 40 to display the composite image generated in Step S112.

### Step S114

The processing circuit 50 receives a registration signal and causes the storage device 30 to store data to be used in an evaluation in the first session. As a result, the data is registered.

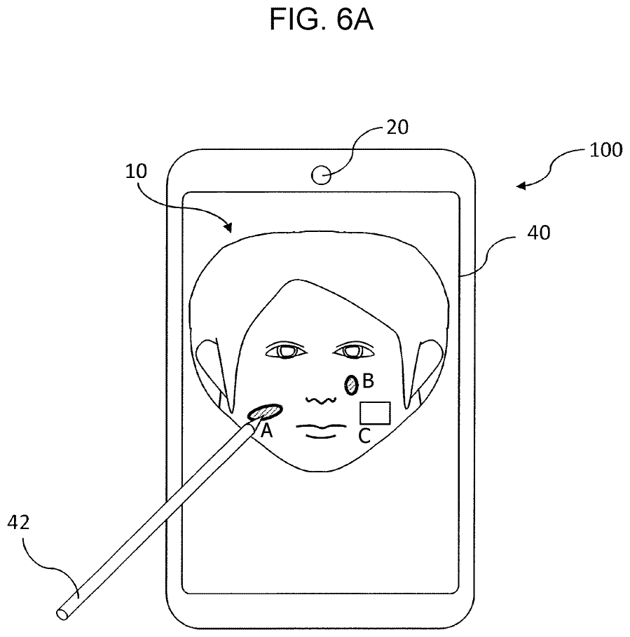

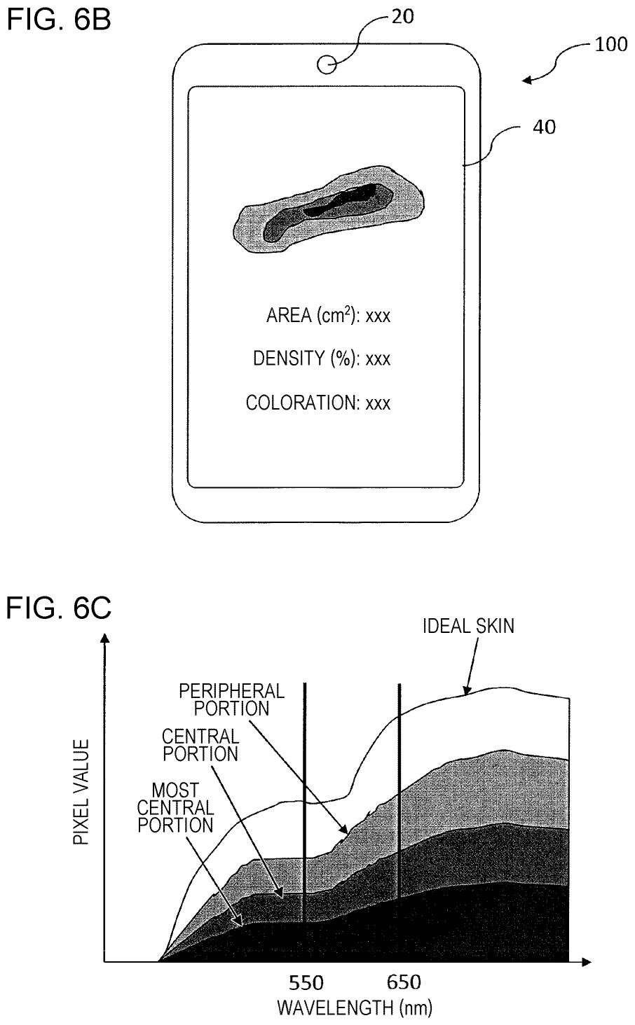

FIGS. 6A and 6B are diagrams for describing the procedure for displaying evaluation results in an evaluation in the first session. As illustrated in FIG. 6A, the display device 40 displays the composite image on which, regarding the registered regions, the edges and labels of the evaluation regions and the perimeter and label of the base region are superposed. As illustrated in FIG. 6A, the user touches, with the touch pen 42, an evaluation region for which the user wants to know evaluation among the registered regions. In the example illustrated in FIG. 6A, the evaluation region A is touched. The processing circuit 50 receives a touch signal, evaluates skin condition in the evaluation region A, and causes the display device 40 to display evaluation results. As illustrated in FIG. 6B, the display device 40 displays a contour diagram of the blemish as well as numerical values for the area, density, and coloration of the blemish as the evaluation results.

The contour diagram of the blemish is a two-dimensional distribution of pixel values for a certain band. Blemishes have melanin pigmentation in the lower part of the epidermis. Since melanin pigmentation absorbs light, pixel values indicating the amounts of reflected light are lower in the evaluation regions than in the base region. The certain band may be a band that is often used to evaluate blemishes, such as a band of a wavelength of 550 nm or 650 nm. The band may have, for example, a wavelength width that is greater than or equal to 1 nm and less than or equal to 10 nm. The contour diagram of the blemish illustrated in FIG. 6B is color-coded into four levels. When the average pixel value in the base region for the certain band is treated as 100%, the four levels are pixel values greater than or equal to 0% and less than 25%, greater than or equal to 25% and less than 50%, greater than or equal to 50% and less than 75%, and greater than or equal to 75% and less than or equal to 100%, and classification is performed using these four levels. The blemish illustrated in FIG. 6B includes the most central, central, and peripheral portions in that order from the inside to the outside. Pixel values in the most central portion are greater than or equal to 0% and less than 25%, pixel values in the central portion are greater than or equal to 25% and less than 50%, and pixel values in the peripheral portion are greater than or equal to 50% and less than 75%. Pixel values around the blemish are greater than or equal to 75% and less than or equal to 100%.

FIG. 6C is a graph illustrating examples of the relationship between pixel value and wavelength, or spectrum, for the ideal skin, the most central portion, the central portion, and the peripheral portion. The spectra for the most central portion, the central portion, and the peripheral portion are those at their outermost boundaries. Pixel values vary with wavelength. For example, the pixel values for the 550 nm wavelength band are lower than the pixel values for the 650 nm wavelength band for the ideal skin, the most central portion, the central portion, and the peripheral portion. The display device 40 may display the graph illustrated in FIG. 6C as an evaluation result.

The area of the blemish is the area of a region enclosed by the edge of the blemish. The density of the blemish is the value obtained by dividing the average pixel value of the evaluation region by the average pixel value of the base region for the certain band. The coloration of the blemish is the ratio of pixel values for any two bands. The coloration may be, for example, the value obtained by dividing the average pixel value of the evaluation region for the 550 nm wavelength band by the average pixel value of the evaluation region for the 650 nm wavelength band. The larger this value, the lighter the yellowish color. Bands at other wavelengths may be selected to evaluate blueness and redness.

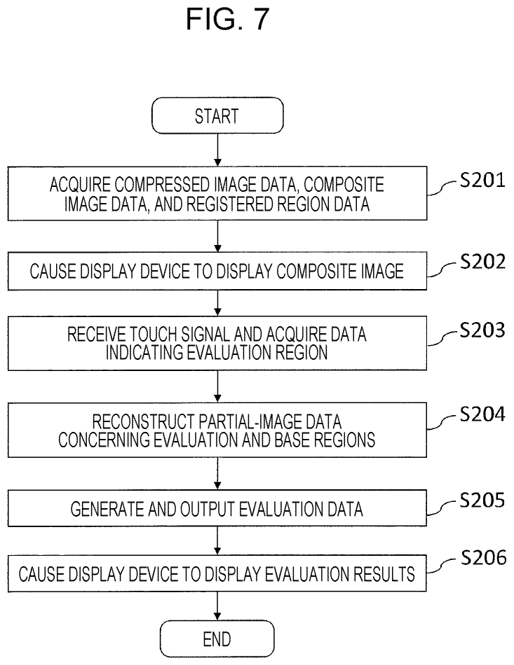

FIG. 7 is a flow chart illustrating an example of an evaluation operation performed by the processing circuit 50 in the procedure described with reference to FIGS. 6A and 6B. The processing circuit 50 performs the operations of the following Steps S201 to S206.

### Step S201

The processing circuit 50 acquires the compressed image data of the left, front, and right sides of the face 10, the data representing the composite image, and the data indicating the registered regions from the storage device 30.

### Step S202

The processing circuit 50 causes the display device 40 to display the composite image on which, regarding the registered regions, the edges and labels of the evaluation regions and the perimeter and label of the base region are superposed.

### Step S203

The processing circuit 50 receives a touch signal and acquires data indicating the evaluation region selected from among the registered regions.

### Step S204

The processing circuit 50 generates partial-image data corresponding to some bands, such as wavelengths of 550 nm and 650 nm, for the evaluation region and base region, for example.

### Step S205

The processing circuit 50 generates and outputs evaluation data on the basis of the partial-image data. The evaluation data represents evaluation results of skin condition in the evaluation region. The evaluation results may be, for example, the contour of the blemish as well as the area, density, and coloration of the blemish. The evaluation results include a comparison result between skin condition in the evaluation region and skin condition in the base region.

### Step S206

The processing circuit 50 causes the display device 40 to display the evaluation results.

In a case where the number of evaluation regions is greater than or equal to two, the processing circuit 50 repeatedly performs the operations of Steps S203 to S206 in accordance with an input to select an evaluation region from the user.

In the evaluation method performed in the first session according to the first embodiment, the evaluation region is determined in accordance with an input from the user. Thus, the processing load can be reduced compared to a method in which the region of a blemish is automatically extracted from the face 10 through image processing. The user determines the evaluation region himself/herself, and thus the user can easily grasp evaluation results of a blemish in a region of the face 10 which the user himself/herself wants to pay attention to. Since skin condition in the evaluation region instead of the entire region of the face 10 is evaluated, faster processing is possible.

### Evaluation Method in Second and Subsequent Sessions

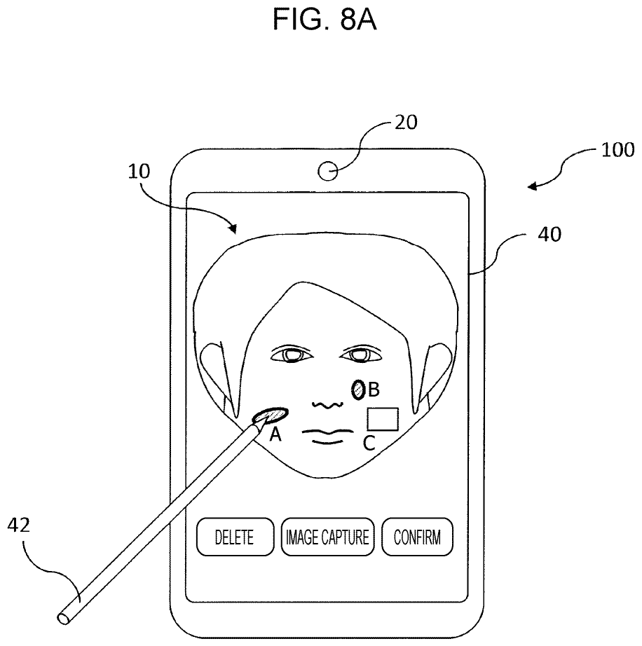

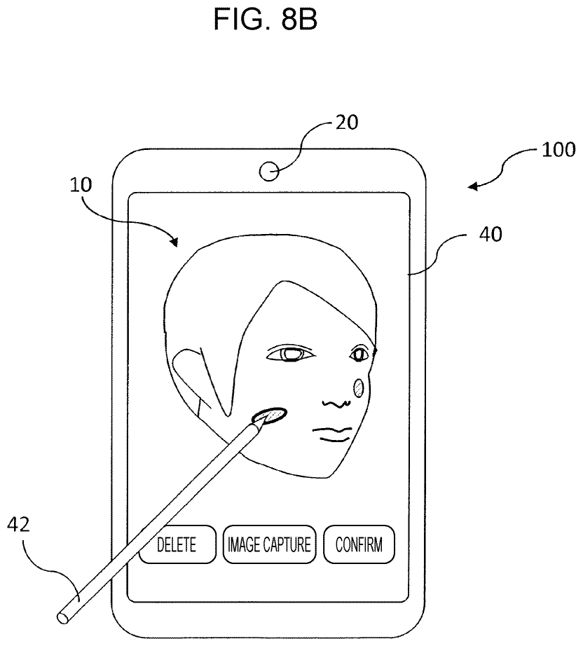

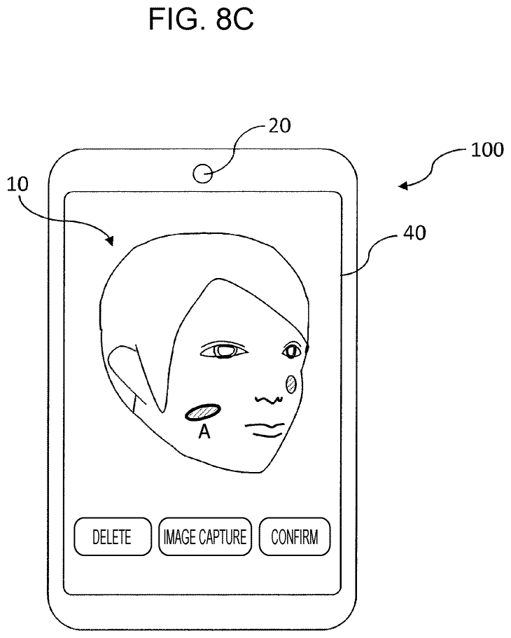

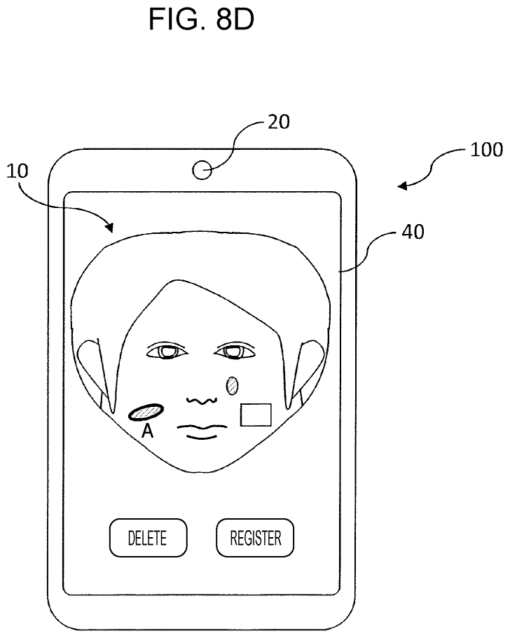

In the following, an evaluation method in the second and subsequent sessions will be described with reference to FIGS. 8A to 11. FIGS. 8A to 8D are diagrams for describing the procedure for registering the current evaluation regions in evaluations in the second and subsequent sessions.

As illustrated in FIG. 8A, the display device 40 displays the composite image on which, regarding the registered regions in the first session, the edges and labels of the evaluation regions and the perimeter and label of the base region are superposed. The processing circuit 50 acquires data indicating the start date and time of the current session that is the second or subsequent session. As illustrated in FIG. 8A, the user touches, with the touch pen 42, an evaluation region for which the user currently wants to know evaluation among the registered regions. In the example illustrated in FIG. 8A, the user selects the evaluation region A on the left side of the face 10 from among the registered regions. The processing circuit 50 receives a touch signal and acquires the data indicating the selected evaluation region.

Next, with the hyperspectral camera 20 of the evaluation apparatus 100 oriented so as to face the face 10 from the front left, the user touches the image capture button displayed by the display device 40 with the touch pen 42. As illustrated in FIG. 8B, the display device 40 displays a captured image of the left side of the face 10. As illustrated in FIG. 8B, the user touches, with the touch pen 42, a region near the center of the blemish for which the user currently wants to know evaluation. The processing circuit 50 receives a touch signal and acquires data indicating the position that is specified with a touch. The processing circuit 50 determines, on the basis of the specified position, the region of the blemish including the specified position to be the same as the selected evaluation region. The above-described facial coordinate system is used for this determination. In the facial coordinate system, in a case where the specified position is inside the selected evaluation region, the region of the blemish can be determined to be the same as the selected evaluation region. Next, the processing circuit 50 extracts the region of the blemish through edge detection, determines the region to be the current evaluation region, and adds the same label as the selected evaluation region in the above description to the current evaluation region. The processing circuit 50 causes the display device 40 to display the edge and label of the current evaluation region as illustrated in FIG. 8C.

In a case where not only the evaluation region A but also the evaluation region B illustrated in FIG. 8A are to be selected, the same procedure described with reference to FIGS. 8A to 8C is repeated for the front of face 10. The number of current evaluation regions is greater than or equal to one. In a case where the current evaluation region is to be confirmed, the user selects the confirm button.

In a case where the user selects the confirm button, the processing circuit 50 receives a confirmation signal and causes the display device 40 to display, as illustrated in FIG. 8D, the composite image on which the edge and label of the current evaluation region are superposed. As illustrated in FIG. 8D, the perimeter of the base region may be superposed on the composite image. In a case where the region of the blemish has increased or decreased, the edge of the current evaluation region does not match the edge of the evaluation region corresponding to the current evaluation region among the registered regions. In a case where the current evaluation region is to be registered, the user selects the register button. The processing circuit 50 receives a registration signal and causes the storage device 30 to store data to be used in the current evaluation. The data include data indicating the start date and time of the current session that is the second or subsequent session, compressed image data of the left side of the current face 10, and data indicating the current evaluation region.

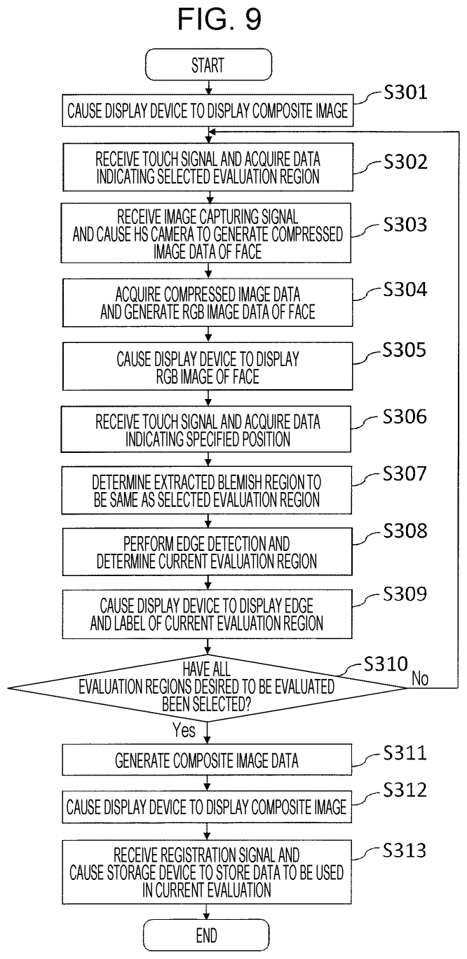

FIG. 9 is a flow chart illustrating an example of an operation performed by the processing circuit 50 in the procedure described with reference to FIGS. 8A to 8D. The processing circuit 50 performs the operations of the following Steps S301 to S313.

### Step S301

The processing circuit 50 causes the display device 40 to display the composite image on which, regarding the registered regions in the first session, the edges and labels of the evaluation regions and the perimeter and label of the base region are superposed. The processing circuit 50 acquires data indicating the start date and time of the current session that is the second or subsequent session.

### Step S302

The processing circuit 50 receives a touch signal and acquires data indicating the evaluation region selected from among the registered regions.

### Steps S303 to S305

Steps S303 to S305 are the same as Steps S102 to S104 illustrated in FIG. 5, respectively.

### Step S306

The processing circuit 50 receives a touch signal and acquires data indicating the specified position.

### Step S307

The processing circuit 50 determines, on the basis of the specified position, the region of the blemish including the specified position to be the same as the selected evaluation region.

### Step S308

The processing circuit 50 performs edge detection to extract the region of the blemish, determines the region to be the current evaluation region, and adds the same label as the selected evaluation region to the current evaluation region.

### Step S309

The processing circuit 50 causes the display device 40 to display the edge and label of the current evaluation region.

### Step S310

The processing circuit 50 determines whether all the evaluation regions desired to be evaluated have been selected from the registered regions. In a case where a confirmation signal is received, the processing circuit 50 can determine that all the evaluation regions desired to be evaluated have been selected. In a case where a confirmation signal is not received within a predetermined time period, the processing circuit 50 can determine that all the evaluation regions desired to be evaluated have not been selected. When Yes in Step S310, the processing circuit 50 performs the operation of Step S311. When No in Step S310, the processing circuit 50 performs the operation of Step S302 again.

### Step S311

The processing circuit 50 generates composite image data obtained by superposing the edge and label of the current evaluation region.

### Step S312

The processing circuit 50 causes the display device 40 to display a composite image based on the composite image data generated in Step S311.

### Step S313

The processing circuit 50 receives a registration signal and causes the storage device 30 to store data to be used in the current evaluation. As a result, the data is registered.

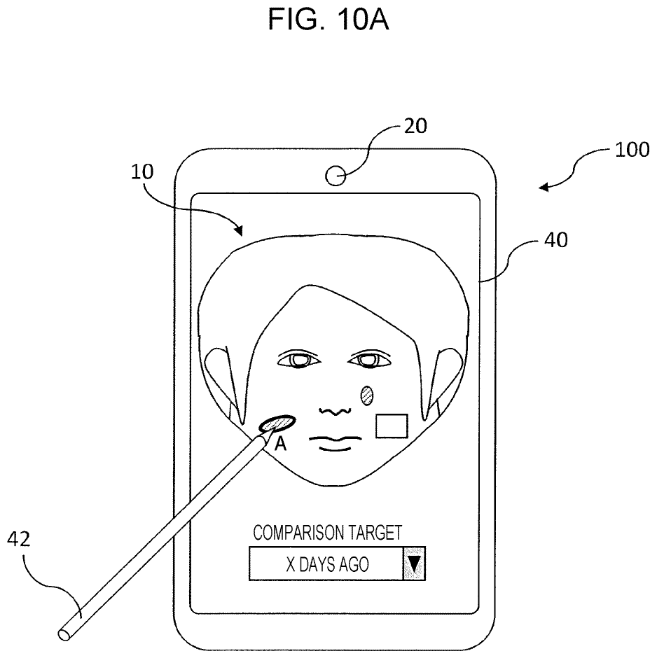

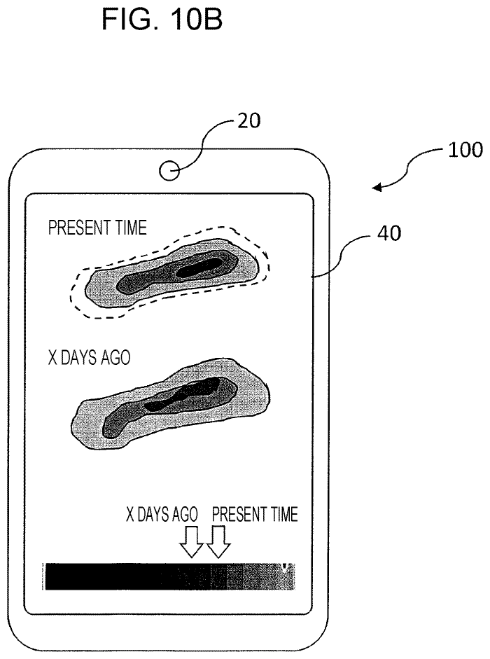

FIGS. 10A and 10B are diagrams for describing the procedure for displaying evaluation results in evaluations in the second and subsequent sessions. As illustrated in FIG. 10A, the display device 40 displays the composite image on which the edge and label of the current evaluation region are superposed. As illustrated in FIG. 10A, the perimeter of the base region may be superposed on the composite image. Furthermore, the display device 40 displays a comparison target as a candidate in a pull-down format. In the example illustrated in FIG. 10A, the user selects the skin condition obtained X days ago as the comparison target. In the case of an evaluation in the second session, “X days ago” corresponds to the first session. In the case of an evaluation in the third or subsequent session, “X days ago” corresponds to any one of the first to previous sessions. The user touches, with the touch pen 42, the current evaluation region among the registered regions, as illustrated in FIG. 10A. The processing circuit 50 receives a touch signal and causes the display device 40 to display evaluation results of the current evaluation region.

As illustrated in FIG. 10B, the display device 40 displays, as the evaluation results, contour diagrams of the blemishes obtained at the present time and X days ago and a bar representing the densities of the blemishes obtained at the present time and X days ago. In the contour diagram of the blemish obtained at the present time, the region enclosed by a dotted line represents the region of the blemish obtained X days ago. By comparing the contour diagrams of the blemishes obtained at the present time and X days ago, the user can see changes in blemish size and density. On the bar for indicating the densities of blemishes, the densities of the blemishes obtained at the present time and X days ago are indicated by arrows. The density of the blemish obtained at the present time has, for a certain band, a value obtained by dividing the average pixel value within the current evaluation region by the average pixel value within the base region obtained X days ago. For this division, not the average pixel value within the base region obtained X days ago but the average pixel value within the base region obtained at the present time may be used. The current imaging environment and the imaging environment X days ago may be such that the average pixel value within the base region obtained at the present time is equal to the average pixel value within the base region obtained X days ago. The display device 40 may display, as evaluation results, numerical values of the area, density, and coloration of the blemish obtained at the present time.

The spectra at all the pixels may be compared between the blemish obtained at the present time and the blemish obtained X days ago using the Spectral Angle Mapper (SAM). In SAM, each pixel has an N-dimensional vector below. The N-dimensional vector is defined by pixel values for N bands included in the target wavelength range. A change in the spectrum of a certain pixel can be checked using the angle formed by the current vector and the vector obtained X days ago at the pixel. In a case where the angle is 0°, the both spectra at the pixel are equal to each other. In a case where the absolute value of the angle is greater than 0°, the both spectra at the pixel are different from each other. By checking the angle formed by the current vector and the vector obtained X days ago at each pixel, changes in spectrum can be obtained as a two-dimensional distribution. In machine learning, learning may be performed using the correspondence between vector orientation and coloration as supervisor data. Through this machine learning, the colorations of the most central, central, and peripheral portions obtained at the present time and X days ago may be determined from the average vector of each of the most central, central, and peripheral portions, and the colorations may be compared between at the present time and X days ago.

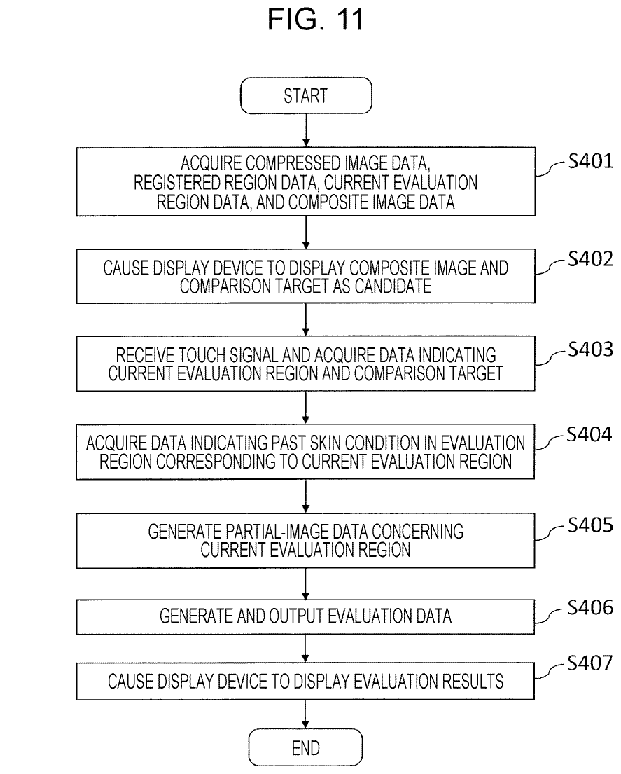

FIG. 11 is a flow chart illustrating an example of an operation performed by the processing circuit 50 in the procedure described with reference to FIGS. 10A and 10B. The processing circuit 50 performs the operations of the following Steps S401 to S407.

### Step S401

The processing circuit 50 acquires, from the storage device 30, the compressed image data of the current face 10, data indicating the registered regions, data indicating the current evaluation region, and data representing the composite image.

### Step S402

The processing circuit 50 causes the display device 40 to display the composite image and a comparison target as a candidate. The edge and label of the current evaluation region are superposed on the composite image.

### Step S403

The processing circuit 50 receives a touch signal and acquires data indicating the current evaluation region and the comparison target.

### Step S404

The processing circuit 50 acquires, on the basis of the comparison target, data indicating past skin condition in the evaluation region corresponding to the current evaluation region from the storage device 30.

### Step S405

The processing circuit 50 generates partial-image data concerning the current evaluation region.

### Step S406

The processing circuit 50 generates and outputs evaluation data on the basis of the partial-image data. The evaluation data represents evaluation results of skin condition in the current evaluation region. The evaluation results include comparison results between the current skin condition and the past skin condition.

### Step S407

The processing circuit 50 causes the display device 40 to display the evaluation results.

With the evaluation method for the second and subsequent sessions according to the first embodiment, it is possible to know the way in which skin condition in an evaluation region changes over time. The evaluation region is determined in accordance with an input from the user, and thus the user can easily grasp evaluation results of a blemish in the region that the user wants to pay attention to on the face 10. Since not skin condition in all the evaluation regions but skin condition in the evaluation region for which the user wants to know evaluation is evaluated among the registered regions, faster processing is possible.

### Data Stored in Storage Device

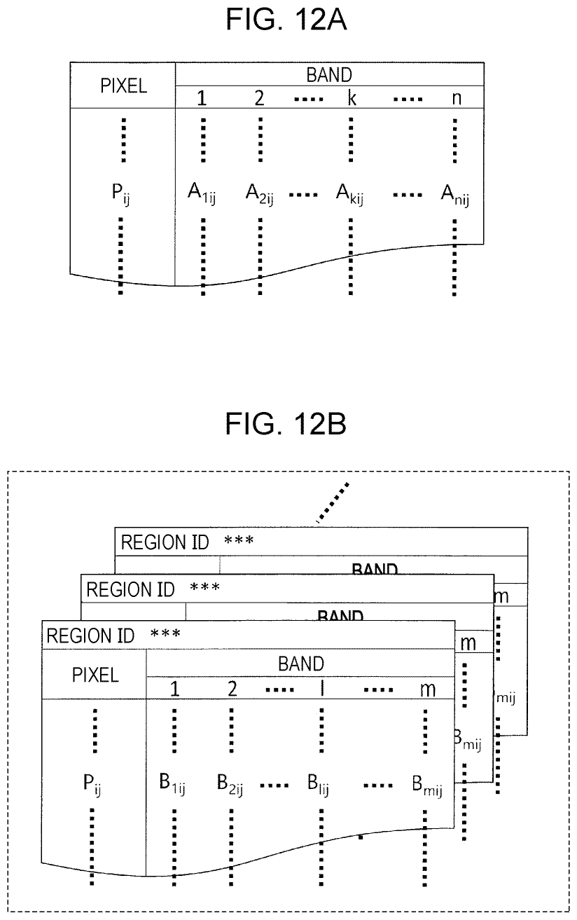

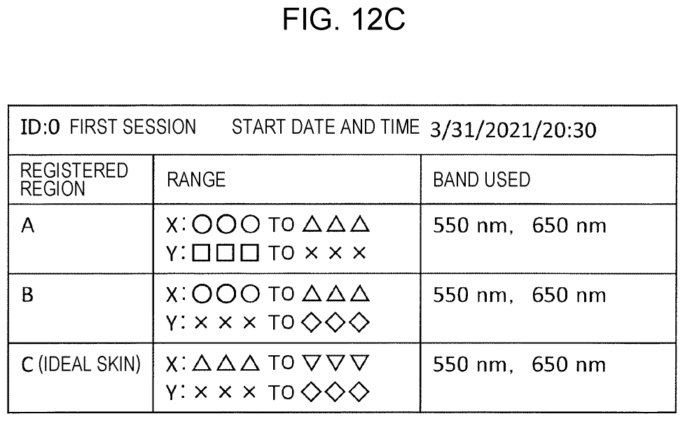

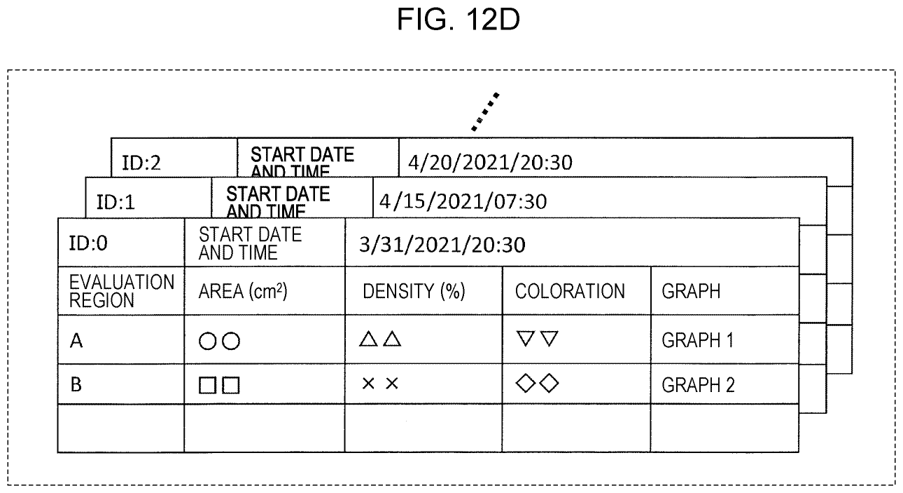

Next, an example of data stored in the storage device 30 will be described with reference to FIGS. 12A to 12D.

FIG. 12A is a diagram schematically illustrating an example of the full-reconstruction table stored in the storage device 30. “Pᵢⱼ” illustrated in FIG. 12A represents the position of a pixel. “Aₖᵢⱼ” illustrated in FIG. 12A represents a luminous transmittance at the pixel Pᵢⱼ in the k-th band. k=1, 2, . . . , n.

FIG. 12B is a diagram schematically illustrating an example of the partial-reconstruction tables stored in the storage device 30. As illustrated in FIG. 12B, a region ID is assigned to each of the evaluation and base regions. “Pᵢⱼ” illustrated in FIG. 12B represents the position of a pixel in each of the evaluation and base regions. “B₁ᵢⱼ” illustrated in FIG. 12B represents a luminous transmittance at the pixel Pᵢⱼ for the 1-th band in each region. l=1, 2, . . . , m. The number of bands included in the partial-reconstruction tables may be equal to or smaller than the number of bands included in the full-reconstruction table.

FIG. 12C is a diagram schematically illustrating an example of a table of the registered regions in the first session and stored in the storage device 30. The table illustrated in FIG. 12C includes information regarding the start date and time, registered region label information, range information, and information regarding bands used. The region label information includes the labels of the evaluation region A, the evaluation region B, and the base region C illustrated in FIG. 4E. The range information includes an X-coordinate range and a Y-coordinate range for each region. The evaluation regions are not actually rectangular in shape but are enclosed by curves. Thus, the range information regarding each evaluation region is not simply a range of X-coordinates and a range of Y-coordinates, but may include, for example, the positions of pixels on the edges in the facial coordinate system. The information regarding bands used includes, for each region, information regarding bands used to check pixel values. In the example illustrated in FIG. 12C, the bands used are the above-described 550 nm and 650 nm wavelength bands.

FIG. 12D is a diagram schematically illustrating an example of tables of evaluation results stored in the storage device 30. The tables illustrated in FIG. 12D include evaluation results obtained in the first session and evaluation results obtained in the second and subsequent sessions. Each table includes information regarding the start date and time, evaluation region label information, area information, density information, coloration information, and graph information. The evaluation region label information includes the evaluation regions A and B. The area information, the density information, and the coloration information include an area numerical value, a density numerical value, and a coloration numerical value for each region. The graph information includes the label of a spectrum graph for each region. Graphs with labels such as “graph 1” and “graph 2” are, for example, graphs as illustrated in FIG. 6C. These graphs are additionally stored in the storage device 30.

### Modification of First Embodiment

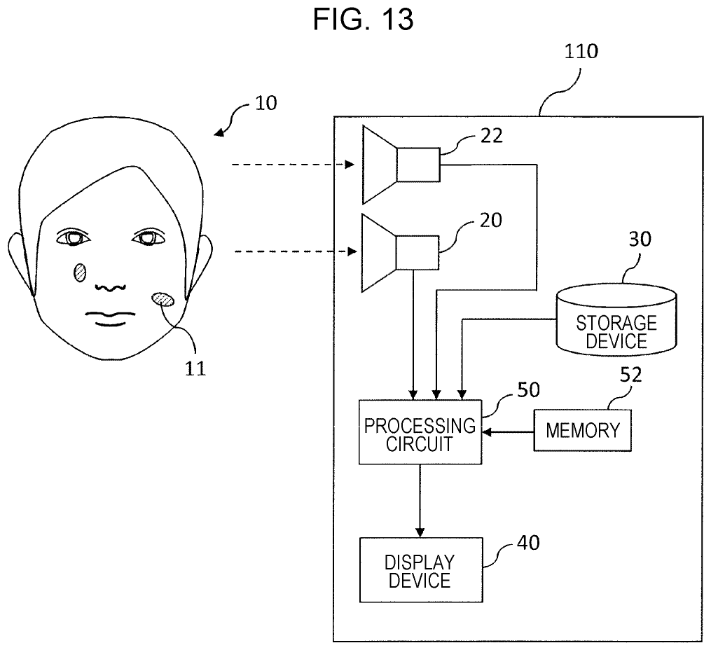

Next, a modification of the evaluation apparatus 100 according to the first embodiment will be described with reference to FIG. 13. FIG. 13 is a diagram schematically illustrating the configuration of an evaluation apparatus 110, which is an exemplary modification of the first embodiment. The evaluation apparatus 110 illustrated in FIG. 13 includes a camera 22 in addition to the configuration illustrated in FIG. 3. The camera 22 is a typical camera that generates, through image capturing, image data for one or more, but no more than three bands. The image data may be, for example, RGB image data or monochrome image data.

In the modification of the first embodiment, the processing circuit 50 performs the following operation instead of Steps S102 and S103 illustrated in FIG. 5 and Steps S303 and S304 illustrated in FIG. 9. The processing circuit 50 causes the camera 22 to generate and output RGB image data of the face 10 and acquires the data from the camera 22. Furthermore, in Step S105 illustrated in FIG. 5 and Step S306 illustrated in FIG. 9, the processing circuit 50 receives a touch signal and not only acquires data indicating the specified position but also causes the hyperspectral camera 20 to generate compressed image data of the face 10 through image capturing. The operation other than those described above is the same as the operation of the processing circuit 50 in the first embodiment.

In the modification of the first embodiment, the RGB image data is not generated from the compressed image data and can be directly generated by the camera 22. Thus, the processing load can be reduced.

In this specification, image data concerning part of the user's body and including information for the four or more bands is also referred to as “first image data”, and image data concerning part of the user's body and including information for the one or more, but no more than three bands is also referred to as “second image data”.

### Second Embodiment

### Evaluation System

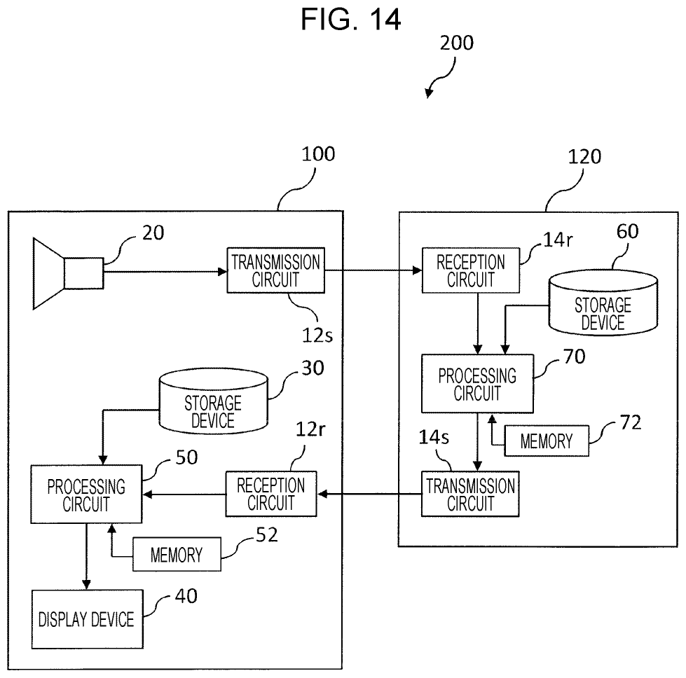

In the first embodiment, the processing circuit 50 included in the evaluation apparatus 100 performs edge detection, generates RGB image data and partial-image data from compressed image data, and generates evaluation data on the basis of the partial-image data. In a case where an external server is connected to the evaluation apparatus 100 via a communication network, a processing circuit included in the external server may perform edge detection, may generate the RGB image data and the partial-image data from the compressed image data, or may generate the evaluation data on the basis of the partial-image data. These operations are assigned to the external server are, for example, in a case where the processing load on the processing circuit 50 included in the evaluation apparatus 100 is desired to be reduced. In this specification, the evaluation apparatus and the server are collectively referred to as an “evaluation system”. In the following, with reference to FIG. 14, an example of an evaluation system according to a second embodiment will be described.

FIG. 14 is a block diagram schematically illustrating an example of an evaluation system 200 according to the second embodiment. As illustrated in FIG. 14, the evaluation system 200 includes the evaluation apparatus 100 and a server 120, which are connected to each other via a wired or wireless communication network. The evaluation apparatus 100 illustrated in FIG. 14 further includes a transmission circuit 12s and a reception circuit 12r in addition to the configuration of the evaluation apparatus 100 illustrated in FIG. 3. The hyperspectral camera 20 outputs compressed image data toward the transmission circuit 12s. The server 120 includes a transmission circuit 14s, a reception circuit 14r, a storage device 60, a processing circuit 70, and a memory 72. The storage device 60 stores a reconstruction table, data indicating registered regions, and evaluation data. The relationship between the processing circuit 70 and the memory 72 in the server 120 is substantially the same as the relationship between the processing circuit 50 and the memory 52 in the evaluation apparatus 100. The evaluation apparatus 100 transmits data to and receives data from the server 120 using the transmission circuit 12s and the reception circuit 12r. The server 120 transmits data to and receives data from the evaluation apparatus 100 using the transmission circuit 14s and the reception circuit 14r. In this specification, the processing circuit 50 included in the evaluation apparatus 100 is referred to as a “first processing circuit 50”, and the processing circuit 70 included in the server 120 is referred to as a “second processing circuit 70”.

### Evaluation Method in First Session

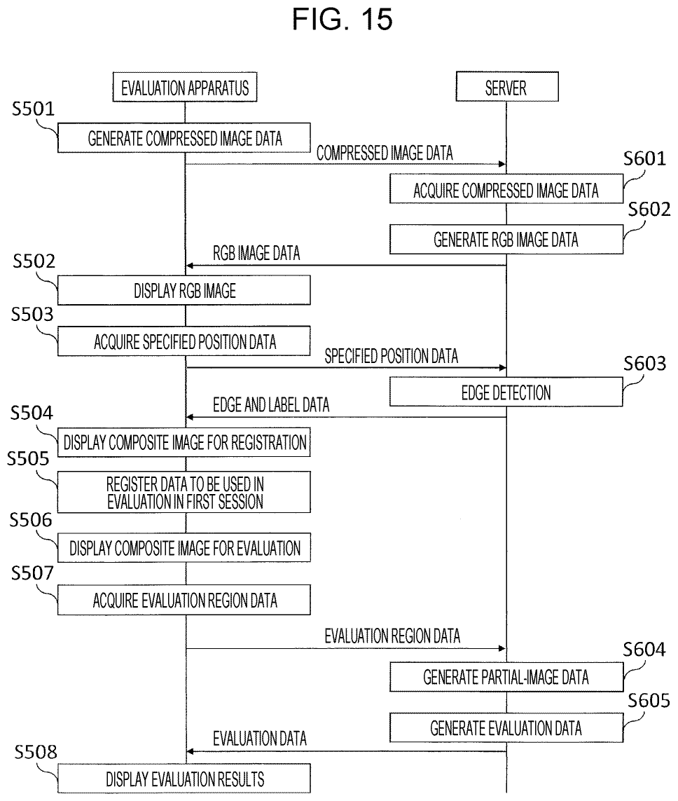

Next, an example of an operation performed between the evaluation apparatus 100 and the server 120 in an evaluation in the first session will be described with reference to FIG. 15. FIG. 15 is a sequence diagram illustrating an operation performed in the first session between the evaluation apparatus 100 and the server 120 according to the second embodiment. The first processing circuit 50 performs the operations of the following Steps S501 to S508. The second processing circuit 70 performs the operations of the following Steps S601 to S605. Each step is described below in chronological order. In the following description, for simplicity, some of the operations of the steps described in the first embodiment are omitted.

### Step S501

The first processing circuit 50 receives an image capturing signal and causes the hyperspectral camera 20 to generate compressed image data of the face 10 of the user. The transmission circuit 12s included in the evaluation apparatus 100 transmits the compressed image data to the reception circuit 14r included in the server 120.

### Step S601

The second processing circuit 70 acquires the compressed image data.

### Step S602

The second processing circuit 70 generates, using the reconstruction table, RGB image data from the compressed image data. The transmission circuit 14s included in the server 120 transmits the RGB image data to the reception circuit 12r included in the evaluation apparatus 100.

### Step S502

The first processing circuit 50 causes the display device 40 to display an RGB image based on the RGB image data.

### Step S503

The first processing circuit 50 receives a touch or long press signal and acquires data indicating the specified position. The transmission circuit 12s included in the evaluation apparatus 100 transmits the data indicating the specified position to the reception circuit 14r included in the server 120.

### Step S603

When a touch signal is received, the second processing circuit 70 extracts the region of a blemish on the basis of the specified position, determines the region to be an evaluation region, and assigns a label to the region. The transmission circuit 14s included in the server 120 transmits data indicating the edge and label of the evaluation region to the reception circuit 12r included in the evaluation apparatus 100.

When a long press signal is received, the second processing circuit 70 determines a rectangular region having a certain area including the position that is specified with a long press to be a base region, and assigns a label to the base region. The transmission circuit 14s included in the server 120 transmits data indicating the perimeter and label of the base region to the reception circuit 12r included in the evaluation apparatus 100.

The operations of Steps S501 to S603 described above are performed every time an image of the face 10 is captured from a different angle.

### Step S504

The first processing circuit 50 causes the display device 40 to display a composite image for registration, the composite image being obtained by connecting the images of the face 10 captured from different angles. On the composite image, the edges and labels of the evaluation regions and the perimeter and label of the base region are superposed to indicate the evaluation regions and the base region.

### Step S505

The first processing circuit 50 receives a registration signal and causes the storage device 30 to store data to be used in an evaluation in the first session. As a result, the data is registered.

### Step S506

The first processing circuit 50 causes the display device 40 to display a composite image for evaluation on which, regarding the registered regions, the edges and labels of evaluation regions and the perimeter and label of the base region are superposed.

### Step S507

The first processing circuit 50 receives a touch signal and acquires data indicating the evaluation region selected from among the registered regions. The transmission circuit 12s included in the evaluation apparatus 100 transmits the data indicating the evaluation region to the reception circuit 14r included in the server 120.

### Step S604

The second processing circuit 70 generates partial-image data concerning the selected evaluation region.

### Step S605

The second processing circuit 70 generates and outputs evaluation data on the basis of the partial-image data. The evaluation data represents evaluation results of skin condition in the selected evaluation region. The second processing circuit 70 causes the storage device 60 to store the evaluation data. The transmission circuit 14s included in the server 120 transmits the evaluation data to the reception circuit 12r included in the evaluation apparatus 100.

### Step S508

The first processing circuit 50 causes the display device 40 to display the evaluation results. The first processing circuit 50 may store the evaluation data into the storage device 30.

### Evaluation Method in Second and Subsequent Sessions

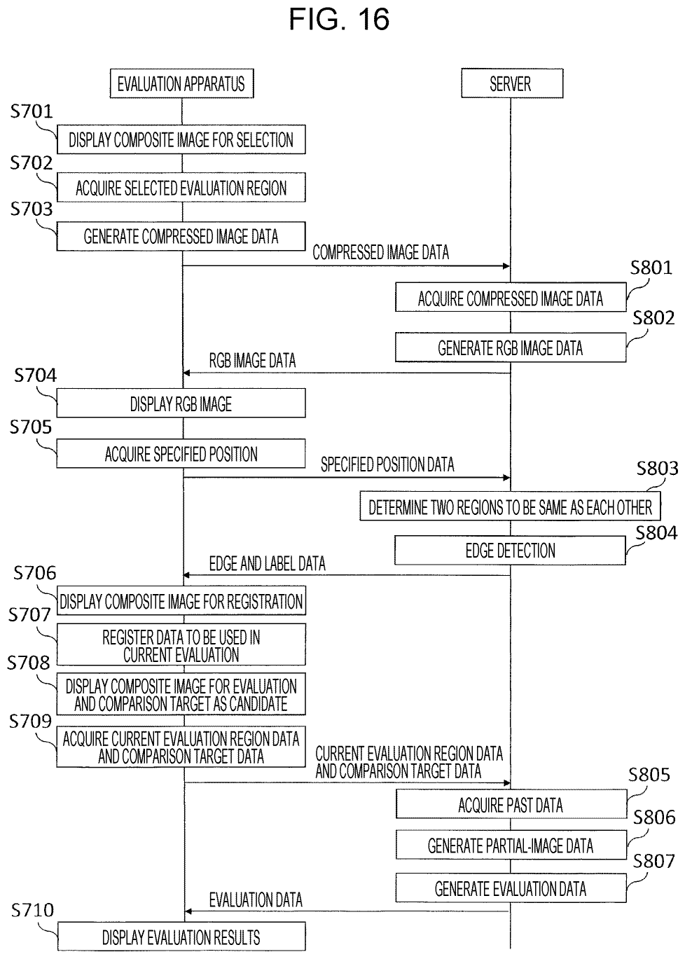

Next, with reference to FIG. 16, an example of an operation performed between the evaluation apparatus 100 and the server 120 in evaluations in the second and subsequent sessions will be described. FIG. 16 is a sequence diagram illustrating an operation performed in the second and subsequent sessions between the evaluation apparatus 100 and the server 120 according to the second embodiment. The first processing circuit 50 performs the operations of the following Steps S701 to S710. The second processing circuit 70 performs the operations of the following Steps S801 to S807. Each step is described below in chronological order. In the following description, for simplicity, some of the operations of the steps described in the first embodiment are omitted.

### Step S701

The first processing circuit 50 causes the display device 40 to display a composite image for selection. On the composite image, the edges and labels of the evaluation regions and the perimeter and label of the base region are superposed to indicate the registered regions in the first session.

### Step S702

The first processing circuit 50 receives a touch signal and acquires data indicating the evaluation region selected from among the registered regions.

### Steps S703 and S704 and Steps S801 and S802

Steps S703 and S704 are the same as Steps S501 and S502 illustrated in FIG. 15, respectively. Steps S801 and S802 are the same as Steps S601 and S602 illustrated in FIG. 15, respectively.

### Step S705

The first processing circuit 50 receives a touch signal and acquires data indicating the specified position. The transmission circuit 12s included in the evaluation apparatus 100 transmits the data indicating the specified position to the reception circuit 14r included in the server 120.

### Step S803

The second processing circuit 70 determines, on the basis of the position that is specified with a touch, the region of a blemish including the specified position to be the same as the selected evaluation region.

### Step S804

The second processing circuit 70 extracts the region of the blemish on the basis of the specified position, determines the region to be the current evaluation region, and assigns, to the region, the same label as the selected evaluation region. The transmission circuit 14s included in the server 120 transmits data indicating the edge and label of the current evaluation region to the reception circuit 12r included in the evaluation apparatus 100.

In a case where the user selects evaluation regions from the registered region, the operations of Steps S701 to S804 described above are performed every time an evaluation region is selected.

### Step S706

The first processing circuit 50 causes the display device 40 to display a composite image for registration on which the edge and label of the current evaluation region are superposed.

### Step S707

The first processing circuit 50 receives a registration signal and causes the storage device 30 to store data to be used in the current evaluation. As a result, the data is registered.

### Step S708

The first processing circuit 50 causes the display device 40 to display a composite image for evaluation on which the edge and label of the current evaluation region are superposed as well as a comparison target as a candidate.

### Step S709

The first processing circuit 50 receives a touch signal and acquires data indicating the current evaluation region and the comparison target. The transmission circuit 12s included in the evaluation apparatus 100 transmits the data indicating the current evaluation region and the comparison target to the reception circuit 14r included in the server 120.

### Step S805

The second processing circuit 70 acquires, on the basis of the data indicating the comparison target, past data indicating past skin condition in an evaluation region corresponding to the current evaluation region from the storage device 60.

### Step S806

The second processing circuit 70 generates partial-image data concerning the current evaluation region.

### Step S807

The second processing circuit 70 generates and outputs evaluation data on the basis of the partial-image data. The evaluation data represents evaluation results of skin condition in the current evaluation region. The second processing circuit 70 stores the evaluation data into the storage device 60. The transmission circuit 14s included in the server 120 transmits the evaluation data to the reception circuit 12r included in the evaluation apparatus 100.

### Step S710

The first processing circuit 50 causes the display device 40 to display the evaluation results. The first processing circuit 50 may store the evaluation data into the storage device 30.

In the evaluation system 200 according to the second embodiment, instead of the first processing circuit 50, the second processing circuit 70 performs edge detection, generates RGB image data and partial-image data from compressed image data, and generates evaluation data on the basis of the partial-image data. Thus, the processing load on the first processing circuit 50 can be reduced. In accordance with the processing performance of the first processing circuit 50, the first processing circuit 50 may perform part of the series of operations performed by the second processing circuit 70.

### Modification of Second Embodiment

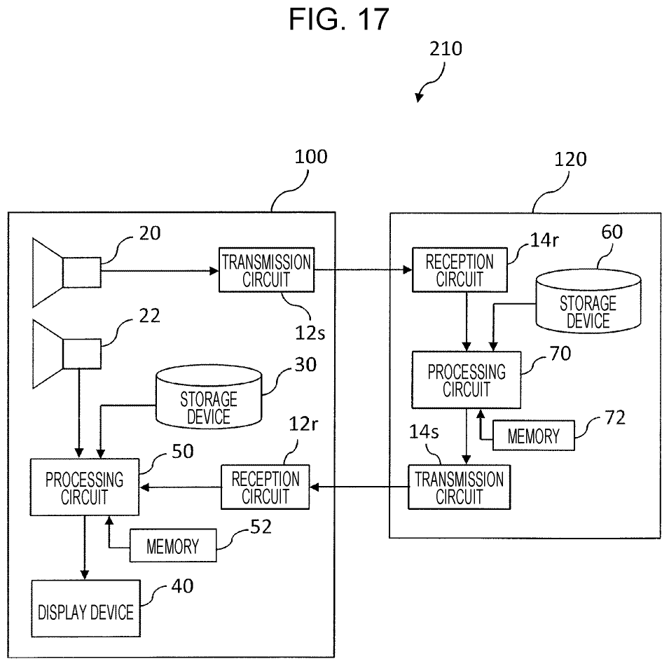

Next, with reference to FIG. 17, a modification, which is an evaluation system 210, according to the second embodiment will be described. FIG. 17 is a diagram schematically illustrating the configuration of the evaluation system 210, which is an exemplary modification of the second embodiment. The evaluation apparatus 100 illustrated in FIG. 17 includes the camera 22 in addition to the configuration of the evaluation apparatus 100 illustrated in FIG. 14. The camera 22 is a camera that generates, through image capturing, image data for one or more, but no more than three bands. The image data may be, for example, RGB image data or monochrome image data.

In the evaluation system 210 according to a modification of the second embodiment, the first processing circuit 50 performs the following operations instead of Steps S501, S601, and S602 illustrated in FIG. 15 and Steps S703, S801, and S802 illustrated in FIG. 16. The first processing circuit 50 causes the camera 22 to generate and output RGB image data of the face 10 and acquires the RGB image data from the camera 22. Furthermore, in Step S503 illustrated in FIG. 15 and Step S705 illustrated in FIG. 16, the first processing circuit 50 receives a touch signal and not only acquires data indicating the specified position but also causes the hyperspectral camera 20 to generate compressed image data of the face 10. The operation other than those described above is the same as the operation of the first processing circuit 50 and the second processing circuit 70 in the second embodiment.

In the evaluation system 210 according to a modification of the second embodiment, the RGB image data is not generated from the compressed image data and can be directly generated by the camera 22. Thus, the processing load on the second processing circuit 70 can be reduced.

The technology according to the present disclosure is applicable, for example, to applications for evaluating skin condition.

1-15. (canceled)

16. A method being performed by a computer, the method comprising:
acquiring compressed image data obtained by compressing image information regarding part of a user's body for four or more wavelength bands into one image;
generating, on the basis of the compressed image, a RGB image including a pixel value corresponding to a wavelength band of red light, a pixel value corresponding to a wavelength band of green light, and a pixel value corresponding to a wavelength band of blue light;
causing a display device to display the RGB image;
determining an evaluation region in the RGB image on the basis of an input from the user;
generating, based on the compressed image data, partial-image data corresponding to at least one wavelength band among the four or more wavelength bands, the partial-image data corresponding to the part of the user's body, the partial-image data including data corresponding to the evaluation region; and
generating, on the basis of the partial-image data, evaluation data indicating an evaluation result of skin condition in the evaluation region.

17. The method according to claim 16, further comprising:
determining a base region located at a different position from the evaluation region in the RGB image, wherein
the evaluation result includes a comparison result between the skin condition in the evaluation region and skin condition in the base region.

18. The method according to claim 16, further comprising:
treating the skin condition in the evaluation region as current skin condition in the evaluation region; and
acquiring data indicating past skin condition in the evaluation region, wherein the evaluation result includes a comparison result between the current skin condition in the evaluation region and the past skin condition in the evaluation region.

19. The method according to claim 16, wherein:
the compressed image data is acquired by imaging the part of the user's body through a filter array,
the filter array has filters arranged two-dimensionally,
transmission spectra of at least two or more filters among the filters are different from each other,
the generating the partial-image data includes generating the partial-image data using at least one reconstruction table corresponding to the at least one wavelength band, and
the reconstruction table indicates a spatial distribution of luminous transmittance of each wavelength band for the filter array in the evaluation region.

20. The method according to claim 16, further comprising causing the display device to display a graphical user interface for the user to specify the evaluation region.

21. The method according to claim 16, wherein the skin condition is a state of a blemish.

22. The method according to claim 16, wherein the at least one wavelength band corresponds to a wavelength of 550 nm or a wavelength of 650 nm.

23. A processing apparatus comprising:
a processor; and
a memory in which a computer program that the processor executes is stored, wherein
the computer program causes the processor to perform:
acquiring compressed image data obtained by compressing image information regarding part of a user's body for four or more wavelength bands into one image,
generating, on the basis of the compressed image, a RGB image including a pixel value corresponding to a wavelength band of red light, a pixel value corresponding to a wavelength band of green light, and a pixel value corresponding to a wavelength band of blue light,
causing a display device to display the RGB image,
determining an evaluation region in the RGB image on the basis of an input from the user,
generating, based on the compressed image data, partial-image data corresponding to at least one wavelength band among the four or more wavelength bands, the partial-image data corresponding to the part of the user's body, the partial-image data including data corresponding to the evaluation region, and
generating, on the basis of the partial-image data, evaluation data indicating an evaluation result of skin condition in the evaluation region.

24. A recording medium, which is non-volatile and computer readable and contains a program for causing a computer to perform a method, the method comprising:
acquiring compressed image data obtained by compressing image information regarding part of a user's body for four or more wavelength bands into one image;
generating, on the basis of the compressed image, a RGB image including a pixel value corresponding to a wavelength band of red light, a pixel value corresponding to a wavelength band of green light, and a pixel value corresponding to a wavelength band of blue light;
causing a display device to display the RGB image;
determining an evaluation region in the RGB image on the basis of an input from the user;
generating, based on the compressed image data, partial-image data corresponding to at least one wavelength band among the four or more wavelength bands, the partial-image data corresponding to the part of the user's body, the partial-image data including data corresponding to the evaluation region; and
generating, on the basis of the partial-image data, evaluation data indicating an evaluation result of skin condition in the evaluation region.

25. A method being performed by a computer, the method comprising:
acquiring image data concerning part of a user's body and including information for four or more wavelength bands;
determining an evaluation region in an image indicating the part of the user's body on the basis of an input from the user;
generating, based on the image data, data indicating current skin condition in the evaluation region;
acquiring data indicating past skin condition in the evaluation region;
generating information including a diagram; and
causing a display devise to display the information, wherein
the diagram includes first areas corresponding to first densities of blemishes in the current skin condition, the first densities being different from each other, the first areas include a second area, and
the diagram indicates densities of blemishes in the past and the current skin conditions in the second area are different from each other.
